(19) United States
(12) Patent Application Publication
(10) Pub. No.: US 2024/0226304 A1
(43) Pub. Date: Jul. 11, 2024

(54) LIPOPEPTIDE FUSION INHIBITORS AS SARS-COV-2 ANTIVIRALS

(71) Applicants: The Trustees of Columbia University in the City of New York, New York, NY (US); Erasmus University Medical Center, Rotterdam (NL); INSERM, Paris (FR)

(72) Inventors: Matteo POROTTO, New York, NY (US); Anne MOSCONA, New York, NY (US); Rik DE SWART, Rotterdam (NL); Rory DE VRIES, Rotterdam (NL); Branka HORVAT, Lyon (FR); Cyrille MATHIEU, Lyon (FR)

(21) Appl. No.: 18/249,058

(22) PCT Filed: Oct. 13, 2021
(86) PCT No.: PCT/US21/54789

§ 371 Date: Apr. 13, 2023

Related U.S. Application Data
(60) Provisional application No. 63/091,915, filed on Oct. 14, 2020.
(60) Provisional application No. 63/107,429, filed on Oct. 29, 2020.
(60) Provisional application No. 63/139,302, filed on Jan. 19, 2021.
(60) Provisional application No. 63/139,306, filed on Jan. 19, 2021.
(60) Provisional application No. 63/144,606, filed on Feb. 2, 2021.
(60) Provisional application No. 63/145,453, filed on Feb. 3, 2021.

(51) Int. Cl. A61K 47/54; A61K 9/00; A61K 38/00; A61P 31/14; C07K 14/005

(52) U.S. Cl. CPC A61K 47/543; A61K 9/0073; A61K 47/554; A61P 31/14; C07K 14/005; A61K 38/00; C12N 2770/20022

(57) ABSTRACT

Described herein is a composition and method of preventing COVID-19 with lipid-peptide fusion antiviral therapy.

This application claims priority to United States Provisional Application Nos. 63/091,915 filed on Oct. 14, 2020; 63/107,429 filed on Oct. 29, 2020; 63/139,302 and 63/139,306 filed on Jan. 19, 2021; 63/144,606 filed on Feb. 2, 2021; and 63/145,453 filed on Feb. 3, 2021, all of which are herein incorporated by reference in their entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT

This invention was made with government support under grants AI114736 and AI121349 awarded by the National Institutes of Health. The government has certain rights in the invention.

### BACKGROUND OF THE INVENTION

Infection by coronaviruses, including the Severe acute respiratory syndrome virus SARS-COV-2 (COVID) virus, requires membrane fusion between the viral envelope and the lung cell membrane. The fusion process is mediated by the virus's envelope glycoprotein, also called spike protein or S. No therapeutic options are currently available for the prophylaxis or treatment of infected individuals. The newly emerged pathogenic virus SARS-CoV-2 (the cause of COVID-19 respiratory disease) represents a worldwide threat to human health and social order. Therefore, given the current pandemic of COVID-19, the development of an effective antiviral therapy against these coronaviruses, especially SARS-CoV-2, is of highest priority not only nationally but also worldwide.

### SUMMARY OF THE INVENTION

In certain aspects, the invention provides a peptide; the C-terminal part of the peptide is “Gly-Ser-Gly-Ser-Cys,” and the N-terminal part of the peptide is selected from SEQ ID NO: 1 and SEQ ID NO:2. In certain aspects, the invention provides a peptide; the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO: 1 and SEQ ID NO:2.

In certain aspects, a SARS lipid-peptide fusion includes a lipid tag, a peptide where the C-terminal part of the peptide is “Gly-Ser-Gly-Ser-Cys,” and the N-terminal part of the peptide is selected from SEQ ID NO:1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2.

In some embodiments, the lipid tag is Cholesterol, Tocopherol, or Palmitate. In some embodiments, the lipid tag is Cholesterol.

In certain aspects, a SARS lipid-peptide fusion inhibitor includes a lipid tag, a spacer, a peptide where the C-terminal part of the peptide is “Gly-Ser-Gly-Ser-Cys,” and the N-terminal part of the peptide is selected from SEQ ID NO:1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2.

In some embodiments, the spacer is a polyethylene glycol (PEG). In some embodiments, the spacer is PEG₄, PEG₁₁, or PEG₂₄. In some embodiments, the lipid tag is Cholesterol, Tocopherol, or Palmitate. In some embodiments, the lipid tag is Cholesterol.

In some embodiments, the SARS lipid-peptide fusion inhibitor has one peptide moiety, one spacer moiety, and one lipid tag. In some embodiments, the inhibitor has two peptide moieties, two spacer moieties, and one lipid tag. The terms “linker” and “spacer” are used interchangeably in the instant application.

In certain aspects, a pharmaceutical composition includes a peptide where the C-terminal part of the peptide is “Gly-Ser-Gly-Ser-Cys,” and the N-terminal part of the peptide is selected from SEQ ID NO: 1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2, and a pharmaceutically acceptable excipient.

In certain aspects, a pharmaceutical composition includes a peptide where the C-terminal part of the peptide is “Gly-Ser-Gly-Ser-Cys,” and the N-terminal part of the peptide is selected from SEQ ID NO: 1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2, a lipid tag, and a pharmaceutically acceptable excipient.

In some embodiments, the lipid tag is Cholesterol, Tocopherol, or Palmitate.

In certain aspects, a pharmaceutical composition includes a peptide where the C-terminal part of the peptide is “Gly-Ser-Gly-Ser-Cys,” and the N-terminal part of the peptide is selected from SEQ ID NO: 1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2, a lipid tag, a spacer, and a pharmaceutically acceptable excipient.

In some embodiments, the spacer is a polyethylene glycol (PEG). In some embodiments, the spacer is PEG₄, PEG₁₁, or PEG₂₄. In some embodiments, the lipid tag is Cholesterol, Tocopherol, or Palmitate.

In some embodiments, the SARS lipid-peptide fusion inhibitor in the pharmaceutical composition has one peptide moiety, one spacer moiety, and one lipid tag. In some embodiments, the inhibitor has two peptide moieties, two spacer moieties, and one lipid tag.

In certain aspects, a SARS-COV-2 (COVID-19) antiviral composition includes a SARS-COV-2 (COVID-19) lipid-peptide fusion inhibitor. The inhibitor further includes two moieties of SEQ ID NO:1, two PEG₄ moieties, one cholesterol tag, and a pharmaceutically acceptable excipient. In some embodiments, each PEG₄ is flanked by a SEQ ID NO: 1 on one end and the cholesterol tag on the other end.

In certain aspects, a SARS-COV-2 (COVID-19) antiviral composition includes a SARS-COV-2 (COVID-19) lipid-peptide fusion inhibitor. The inhibitor further includes one moiety of SEQ ID NO:1, one PEG₄ moiety, one cholesterol tag, and a pharmaceutically acceptable excipient. In some embodiments, the PEG₄ is flanked by a SEQ ID NO: 1 on one end and the cholesterol tag on the other end.

In certain aspects, the invention provides a method of preventing COVID-19 that includes administering to a subject in need an antiviral pharmaceutical composition. The pharmaceutical composition includes a peptide where the C-terminal part of the peptide is “Gly-Ser-Gly-Ser-Cys,” and the N-terminal part of the peptide is selected from SEQ ID NO:1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO: 1 and SEQ ID NO:2, a lipid tag, a spacer, and a pharmaceutically acceptable excipient.

In some embodiments, the lipid tag is Cholesterol, Tocopherol, or Palmitate.

In certain aspects, the invention provides a method of preventing COVID-19 that includes administering to a subject in need an antiviral pharmaceutical composition. The pharmaceutical composition includes a SARS-COV-2 (COVID-19) lipid-peptide fusion inhibitor, which further includes two moieties of SEQ ID NO:1, two PEG₄ moieties, one cholesterol tag, and a pharmaceutically acceptable excipient, wherein each PEG₄ is flanked by SEQ ID NO: 1 on one end and cholesterol on the other end.

In certain aspects, the invention provides a method of preventing COVID-19 that includes administering to a subject in need an antiviral pharmaceutical composition. The pharmaceutical composition includes a SARS-COV-2 (COVID-19) lipid-peptide fusion inhibitor, which further includes one moiety of SEQ ID NO:1, one PEG₂₄ moiety, one cholesterol tag, and a pharmaceutically acceptable excipient, wherein the PEG₂₄ is flanked by SEQ ID NO: 1 on one end and cholesterol on the other end.

In some embodiments, the antiviral pharmaceutical composition is administered per airway or subcutaneously. In some embodiments, the antiviral pharmaceutical composition is administered intranasally. In some embodiments, the antiviral pharmaceutical composition is administered as nasal drops or a spray. In some embodiments, the antiviral pharmaceutical composition is administered as nasal powder.

In some embodiments, the antiviral pharmaceutical composition is administered to the subject at least two times. In some embodiments, at least one administration occurs before the subject is exposed to SARS-COV-2. In some embodiments, all administrations occur before the subject is exposed to SARS-COV-2. In some embodiments, the antiviral pharmaceutical composition is administered daily.

In some embodiments, the antiviral pharmaceutical composition is administered to the subject once. In some embodiments, the administration occurs before the subject is exposed to SARS-COV-2.

In some embodiments, the antiviral pharmaceutical composition is administered to the subject in need thereof with one or more additional antiviral substances. In some embodiments, at least one additional antiviral substance targets a different aspect of SARS-CoV-2 life cycle than SARS_{HRC} peptides.

In some embodiments, the peptide reaches biologically effective concentrations both in upper and lower respiratory tract of the subject. In some embodiments, the peptide reaches biologically effective concentrations in the lungs of the subject. In some embodiments, the peptide reaches biologically effective concentrations in the blood of the subject.

In some embodiments, the method prevents COVID-19 that would have been caused by SARS-COV-2 virions that comprise a Spike protein, wherein the sequence of the Spike protein differs from SEQ ID No:3. In some embodiments, the SARS-COV-2 is selected from the group consisting of SARS-COV-2 S247R, SARS-COV-2 D614G, SARS-COV-2 S943P, and SARS-COV-2 D839Y. In some other embodiments, the SARS-COV-2 is selected from the group consisting of SARS-COV-2 alpha beta, gamma, delta, and lambda variants.

In certain aspects, the invention provides a method of reducing the risk of a SARS-COV-2 infecting a cell in a subject. The method includes administering an effective amount of a SARS-COV-2 (COVID-19) antiviral composition to inhibit SARS-COV-2 infection of a cell. The SARS-COV-2 (COVID-19) antiviral composition includes a SARS-CoV-2 (COVID-19) lipid-peptide fusion inhibitor comprising two moieties of SEQ ID NO:1, two PEG₄ moieties, one cholesterol tag, and a pharmaceutically acceptable excipient. Each PEG₄ can be flanked by a SEQ ID NO: 1 on one end and the cholesterol tag on the other end. Alternatively, The SARS-COV-2 (COVID-19) antiviral composition includes a SARS-COV-2 (COVID-19) lipid-peptide fusion inhibitor comprising one moiety of SEQ ID NO:1, one PEG₂₄ moiety, one cholesterol tag, and a pharmaceutically acceptable excipient. PEG₂₄ can be flanked by a SEQ ID NO: 1 on one end and the cholesterol on the other end.

In certain aspects wherein the SARS-COV-2 (COVID-19) antiviral composition includes a SARS-COV-2 (COVID-19) lipid-peptide fusion inhibitor comprising two moieties of SEQ ID NO:1, two PEG₄ moieties, one cholesterol tag, and a pharmaceutically acceptable excipient, intranasal administration thereof results in an equivalent level of SARS-COV-2 (COVID-19) lipid-peptide fusion inhibitor in the turbinate and in the lungs of the subject by 1 hour after administration. Two levels are equivalent if both levels, e.g., of concentration of lipid-peptide fusion inhibitor, are of the same order of magnitude, or wherein one level is within 25% of the level of the other, or wherein one level is within 50% of the level of the other. In some embodiments, equivalent levels of the SARS-COV-2 (COVID-19) lipid-peptide fusion inhibitor are maintained in both the lungs and the turbinate of the subject up to 8 hours after intranasal administration. In some embodiments, equivalent levels of the SARS-CoV-2 (COVID-19) lipid-peptide fusion inhibitor are maintained in both the lungs and the turbinates of the subject up to 24 hours after intranasal administration. In some embodiments, equivalent levels of the SARS-COV-2 (COVID-19) lipid-peptide fusion inhibitor are maintained in both the lungs and the turbinate of the subject up to 48 hours after intranasal administration.

In certain aspects, the invention provides a method of reducing the risk of COVID-19 in a subject. The method includes administering an effective amount of a SARS-CoV-2 (COVID-19) antiviral composition to inhibit SARS-COV-2 infection of a cell. The SARS-COV-2 (COVID-19) antiviral composition includes a SARS-COV-2 (COVID-19) lipid-peptide fusion inhibitor comprising two moieties of SEQ ID NO: 1, two PEG₄ moieties, one cholesterol tag, and a pharmaceutically acceptable excipient. Each PEG₄ can be flanked by a SEQ ID NO: 1 on one end and the cholesterol tag on the other end. Alternatively, The SARS-COV-2 (COVID-19) antiviral composition includes a SARS-COV-2 (COVID-19) lipid-peptide fusion inhibitor comprising one moiety of SEQ ID NO:1, one PEG₂₄ moiety, one cholesterol tag, and a pharmaceutically acceptable excipient. PEG₂₄ can be flanked by a SEQ ID NO: 1 on one end and the cholesterol on the other end.

In certain aspects, the invention provides a method of reducing the risk of death from COVID-19 in a subject. The method includes administering an effective amount of a SARS-COV-2 (COVID-19) antiviral composition to inhibit SARS-COV-2 infection of a cell. The SARS-COV-2 (COVID-19) antiviral composition includes a SARS-COV-2 (COVID-19) lipid-peptide fusion inhibitor comprising two moieties of SEQ ID NO:1, two PEG₄ moieties, one cholesterol tag, and a pharmaceutically acceptable excipient. Each PEG₄ can be flanked by a SEQ ID NO: 1 on one end and the cholesterol tag on the other end. Alternatively, The SARS-COV-2 (COVID-19) antiviral composition includes a SARS-COV-2 (COVID-19) lipid-peptide fusion inhibitor comprising one moiety of SEQ ID NO: 1, one PEG₂₄ moiety, one cholesterol tag, and a pharmaceutically acceptable excipient. PEG₂₄ can be flanked by a SEQ ID NO: 1 on one end and the cholesterol on the other end.

In some embodiments, the method prevents COVID-19 that would have been caused by SARS-COV-2 virions that comprise a Spike protein, wherein the sequence of the Spike protein differs from SEQ ID NO:3. In some embodiments, the SARS-COV-2 is selected from the group consisting of SARS-COV-2 S247R, SARS-COV-2 D614G, SARS-CoV-2 S943P, and SARS-COV-2 D839Y. In some other embodiments, the SARS-COV-2 is selected from the group consisting of SARS-COV-2 B alpha, beta, gamma, delta, and lambda variants.

### BRIEF DESCRIPTION OF FIGURES

This application contains at least one drawing executed in color.

FIG. 1: SARS-COV-2 spike (S) glycoprotein domain architecture and structure.

FIG. 2: Infection and Cell-entry by coronaviruses.

FIG. 3: Lipid modified HRC peptides block both early and latent coronavirus viral entry.

FIG. 4: Crystal structure of HRC and HRN of the SARS-COV-2 S protein.

FIG. 5: Sequence of the SARS and SARSMod peptides.

FIGS. 6A-C: Peptide-lipid conjugates that inhibit SARS-COV-2 spike (S)-mediated fusion. (A) The functional domains of SARS-COV-2 S protein. (B) Sequence of the peptides that derive from the HRC domain of SARS-COV-2 S. (C) Monomeric and dimeric forms of lipid tagged SARS-COV-2 inhibitory peptides.

FIGS. 7A-E: Identities of the conjugates were verified by MALDI-TOF MS. (A) SARS_{HRC}-PEG₄-chol. (B) [SARS_{HRC}-PEG₄]₂-chol. (C) [SARS_{HRC}]₂-PEG₁₁. (D) SARS_{HRC}-chol. (E) SARS_{HRC}-PEG₂₄-chol.

FIGS. 8A-C: In vitro potency of different SARS lipid-peptide fusions. (A) Cell-cell fusion assays with different inhibitory peptides. (B) Fusion inhibitory activity of [SARS_{HRC}—PEG₄]₂-chol peptide against SARS-COV-2 variants, MERS-COV-2, and SARS-COV. (C) Fusion inhibitory activity of [SARS_{HRC}-PEG₄]₂-chol peptide against additional, recently emerged SARS-COV-2 variants, MERS-COV-2, and SARS-COV.

FIGS. 9A-B: Addition of cell penetrating peptide sequence does not increase the antiviral activity of [SARS_{HRC}-PEG₄]₂-chol. (A) VeroE6 cells. (B) VeroE6-TMPRSS2 cells.

FIGS. 10A-B: Models for mechanism of virus-host cell membrane fusion. (A) Proposed model of interactions between S on the viral envelope and Ace2 on the host cell membrane leading to membrane fusion. (B) Proposed anchoring of the dimeric lipopeptide in the host cell membrane and interactions with the viral S protein to inhibit S-mediated fusion.

FIGS. 11A-C: Design and specificity of SARS-COV-2 inhibition by [SARS_{HRC}-PEG₄]₂-chol. (A) Chemical structure of [SARS_{HRC}-PEG₄]₂-chol. (B) [SARS_{HRC}-PEG₄]₂-chol proved specific (C) Sequences of respective peptides evaluated in FIG. 11B.

FIGS. 12A-E: In vivo biodistribution assessment. (A, B) Administration (SQ) of with [SARS_{HRC}-PEG₄]₂-chol and SARS_{HRC}-PEG₂₄ (C, D) Intranasal administration. (E) Experimental design of biodistribution experiment in hACE2 transgenic mice.

FIG. 13: Lung sections of [SARS_{HRC}-PEG₄]₂-chol-treated (or vehicle-treated) mice at 1, 8, 24 hours post-inoculation (HPI). (A) lung tile scans, scale bar=500 μm (B), 40× images, scale bar=50 μm (C) Antibody specificity test.

FIGS. 14A-B: In vivo biodistribution assessment.

FIG. 15: Ex vivo cytotoxicity assessment.

FIGS. 16A-C: Inhibition of infectious SARS-COV-2 entry by [SARS_{HRC}-PEG₄]₂-chol and [HPIV3_{HRC}-PEG₄]₂-chol peptides. (A) DMSO-formulated stocks (B) Sucrose-formulated stocks (C) Data shown in A and B.

FIGS. 17A-B and 18A-B: Potency of inhibitory lipopeptides (FIPs), monoclonal antibodies (mAbs) or post-vaccination sera against entry of wt SARS-COV-2 and variants of concern (VOC). Tests were performed in VeroE6-TMPRSS2 cells (A) and Calu3 cells (B).

FIGS. 19A-G: Inhibition of wt SARS-COV-2 and VOC entry by fusion inhibitory peptides (FIPs), monoclonal antibodies (mAbs) or post-vaccination sera. Percentage entry inhibition in VeroE6-TMPRSS2 cells is shown for increasing concentrations of FIPs (A), mAbs (B-D) or increasing dilutions of post-vaccination sera (E-G).

FIGS. 20A-B: Fusion inhibitory activity of [SARS_{HRC}-PEG₄]₂-chol peptide against emerging SARS-COV-2 S variants. (A) β-galactosidase complementation assay. (B) Percent inhibition was calculated.

FIGS. 21A-J: [SARS_{HRC}-PEG₄]₂-chol prevents SARS-COV-2 transmission in vivo. (A) Experimental design. (B) Viral loads detected in throat (C) Viral loads detected in nose (D) Comparison of AUC (E) Viral loads detected in throat swabs by live virus isolation on VeroE6. (F) Correlation between viral loads in the throat as detected via RT-qPCR and live virus isolation. (G) Presence of anti-S antibodies (H) Presence of anti-N antibodies (I)Presence of neutralizing antibodies determined in a live virus neutralization assay. (J). Direct inoculation of peptide-treated or mock-treated animals with SARS-COV-2.

FIGS. 22A-D: In vitro potency of peptide stocks used in ferrets. (A) DMSO-formulated stocks on VeroE6 (B) DMSO-formulated stocks on VeroE6-TMPRSS (C) Sucrose-formulated stocks on VeroE6 (D) Sucrose-formulated stocks on VeroE6-TMPRSS.

FIGS. 23A-B: Challenge infection of previously peptide-treated and mock-treated animals with SARS-COV-2. (A) Viral loads in throat swabs were determined on a daily basis by RT-qPCR up to 7 days post inoculation. (B) Area under the curves (AUC) demonstrate that the total genome load slightly decreases corresponding to the challenge dose.

FIGS. 24A-F: A single dose of [SARS_{HRC}-PEG₄]₂-chol provides protection against SARS-COV-2 transmission in vivo. (A) Experimental Design. (B) Viral loads detected in throat (C) Viral loads detected in nose (D) Comparison of the area under the curve (AUC) from genome loads reported in B for [HPIV3_{HRC}-PEG₄]₂-chol-treated and [SARS_{HRC}-PEG₄]₂-chol-treated sentinels. (E) Viral loads detected in throat swabs by live virus isolation on VeroE6. (F) Correlation between viral loads in throat determined by RT-qPCR or infectious virus isolation.

FIGS. 25A-B: Weight loss in control- and peptide-treated ferrets is not significantly different. (A) Body weights of ferrets over time with DMSO-formulated peptides (B) Body weights of ferrets over time with sucrose-formulated peptides.

FIG. 26. In vivo efficacy of SARS peptides in transgenic mice expressing the human ACE2 receptor: the surviving animals developed neutralizing sera.

### DETAILED DESCRIPTION OF FIGURES

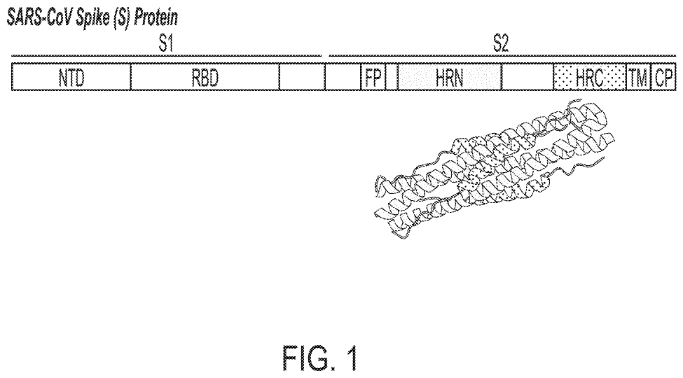

FIG. 1: SARS-COV-2 spike (S) glycoprotein domain architecture and structure. A simplified schematic diagram of SARS-COV-2 S is shown. The N-terminal domain (NTD), receptor-binding domain (RBD), fusion peptide (FP), N-terminal heptad repeat (HRN), C-terminal heptad repeat (HRC), transmembrane (TM), and cytoplasmic tail (CP) domains are depicted. Repeat sections HRN and HRC at either end recognize each other, and snap together to form the folded structure. Fusion inhibitory peptides bind to the repeat section and prevent formation of the folded structure, therefore blocking viral fusion and entry.

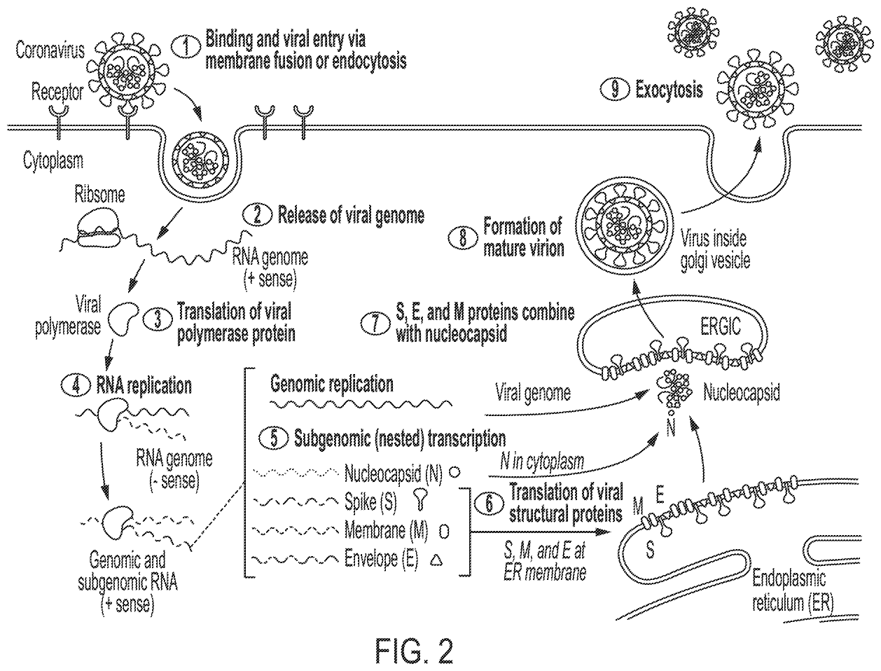

FIG. 2: Infection and Cell-entry by coronaviruses.

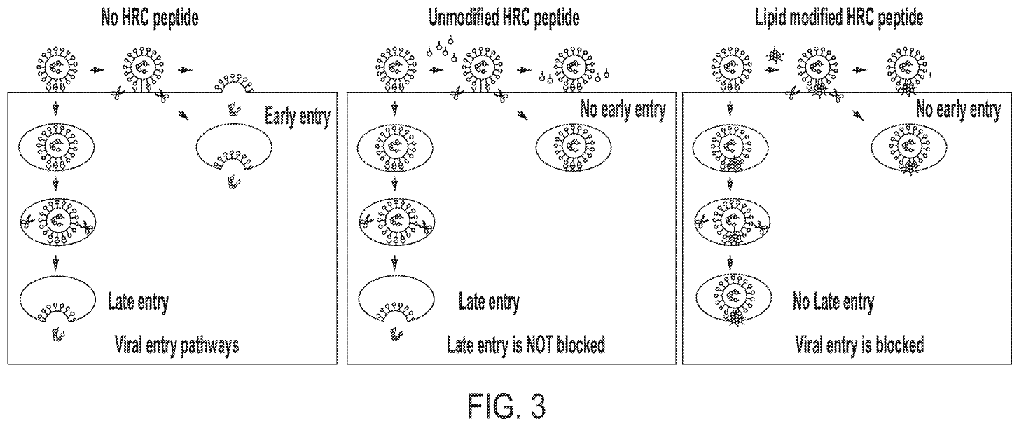

FIG. 3: Lipid modified HRC peptides block both early and latent coronavirus viral entry. This is a schematic representation of results obtained using our lipid-conjugated MERS-derived peptides. Figure from Park and Gallagher, Lipidation increases antiviral activities of coronavirus fusion-inhibiting peptides, Virology 2017; 511, 9-18 at Graphic Abstract.

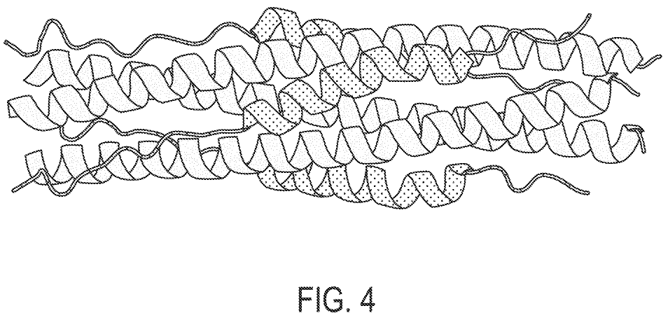

FIG. 4: Crystal structure of the 6HB assembly formed by the HRC (red) and HRN (blue) domains of the SARS-COV-2 S protein (PDB 6LXT). In HRC, note central helix and extended segments on either side.

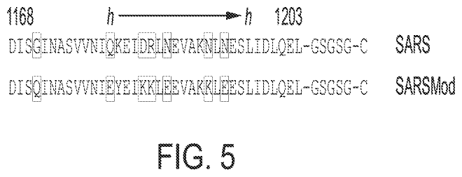

FIG. 5: Sequence of the HRC domain of the SARS-COV-2 S protein (top), with numbering shown at each end, as represented in the peptide SARS. The two “h” symbols indicate the boundaries of the helical segment. The peptide SARSMod contains seven α-amino acid residue changes as compared to the peptide SARS.

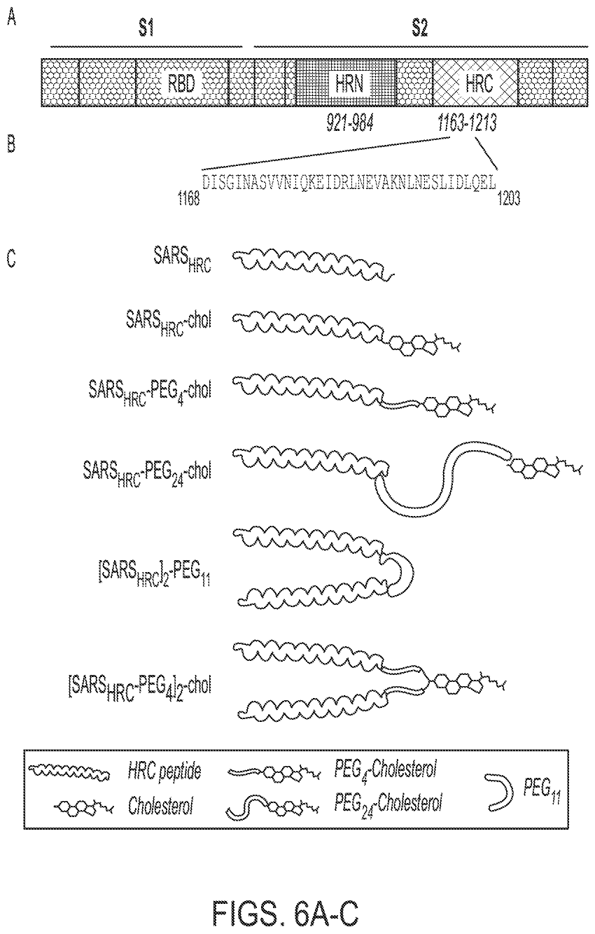

FIGS. 6A-B: Peptide-lipid conjugates that inhibit SARS-COV-2 spike (S)-mediated fusion. (A) The functional domains of SARS-COV-2 S protein: receptor-binding domain (RBD) and heptad repeats (HRN and HRC) are indicated. (B) Sequence of the peptides that derive from the HRC domain of SARS-COV-2 S. (C) Monomeric and dimeric forms of lipid tagged SARS-COV-2 inhibitory peptides that were assessed in cell-cell fusion assays.

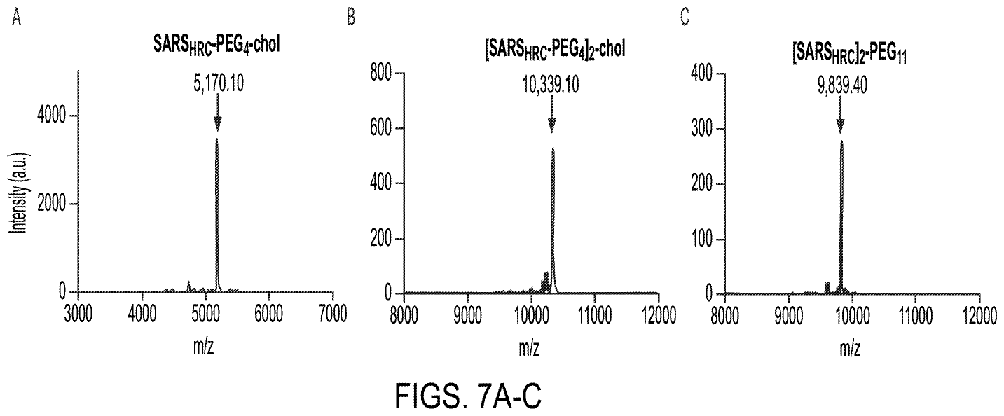

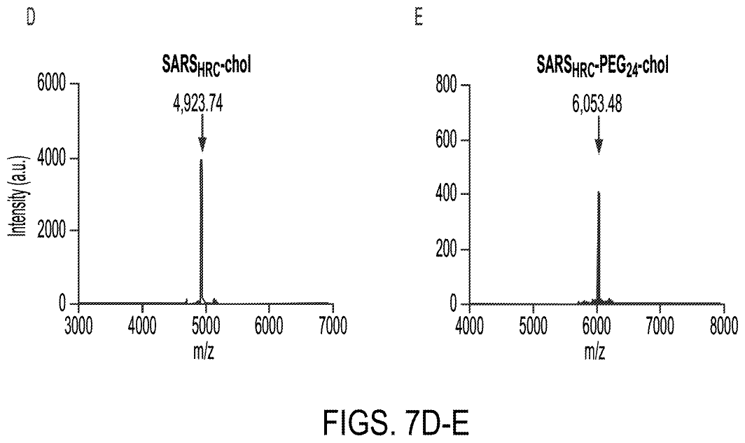

FIGS. 7A-E: Identities of the conjugates were verified by MALDI-TOF MS. (A) MALDI of SARS_{HRC}-PEG₄-chol. Theoretical: 5170.8 Da; observed 5170.1 Da. (B) MALDI of [SARS_{HRC}-PEG₄]₂-chol Theoretical m/z: 10,335.4 Da; observed 10,339.10 Da. (C) MALDI of [SARS_{HRC}]₂-PEG₁₁. Theoretical m/z: 9841.0 Da; observed m/z: 9,839.40 Da. (D) MALDI of SARS_{HRC}-chol. Theoretical m/z: 4923.64 Da; observed 4923.74 Da. (E) MALDI of SARS_{HRC}-PEG₂₄-chol. Theoretical m/z: 6051.31 Da; observed 6053.48 Da.

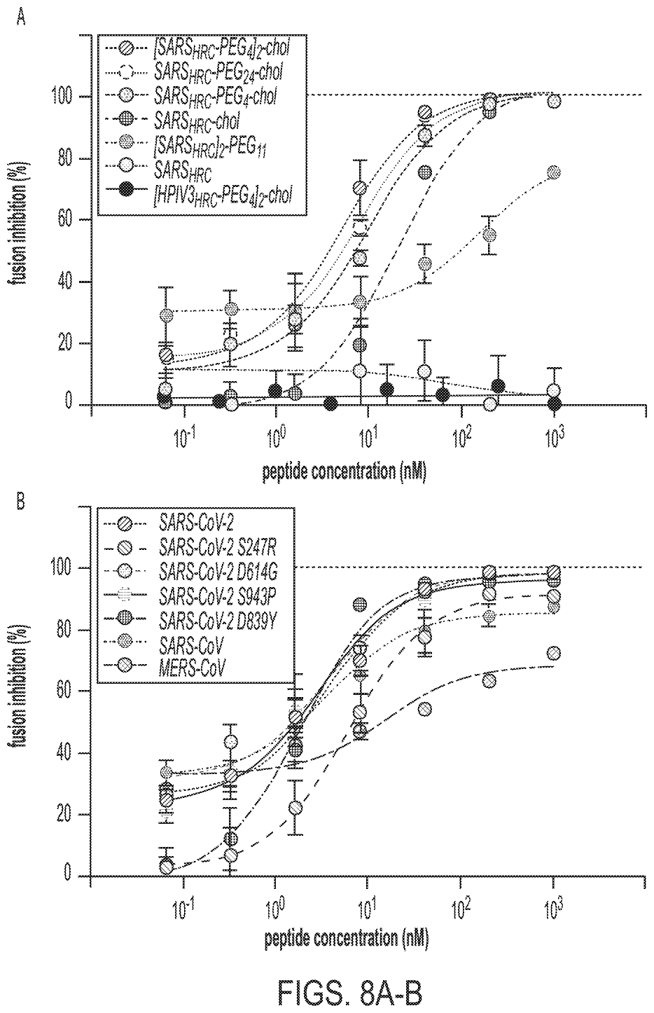

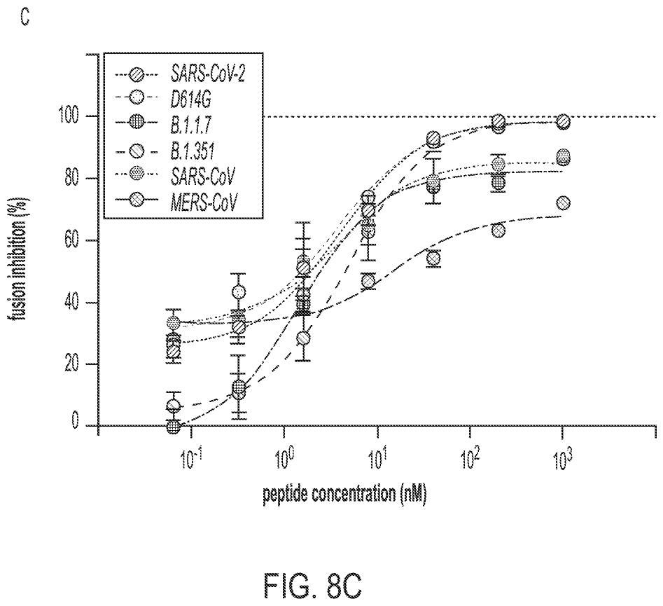

FIGS. 8A-C: In vitro potency of different SARS lipid-peptide fusions. (A) Cell-cell fusion assays with different inhibitory peptides. The percentage inhibition is shown for six different SARS-COV-2-specific peptides and a control HPIV3-specific peptide at increasing concentrations. Percent inhibition was calculated as the ratio of the relative luminescence units in the presence of a specific concentration of inhibitor and the relative luminescence units in the absence of inhibitor, corrected for background luminescence. % inhibition=100×[1−(luminescence at X−background)/(luminescence in absence of inhibitor−background)]. The difference between the results for [SARS_{HRC}-PEG₄]₂-chol and SARS_{HRC}-PEG₄-chol lipopeptides was statistically significant (Two-way ANOVA, p<0.0001). (B) Fusion inhibitory activity of [SARS_{HRC}-PEG₄]₂-chol peptide against SARS-CoV-2 variants (SARS-COV-2 S247R, SARS-COV-2 D614G, SARS-COV-2 S943P, and SARS-COV-2 D839Y), MERS-COV-2, and SARS-COV. (C) Fusion inhibitory activity of [SARS_{HRC}-PEG₄]₂-chol peptide against additional, recently emerged SARS-COV-2 variants (SARS-COV-2 D614G, SARS-COV-2 alpha (B 1.1.7), and SARS-COV-2 beta (B 1.351), MERS-COV-2, and SARS-COV. Data in (A, B, and C) are means±standard error of the mean (SEM) from three separate experiments with the curve representing a four-parameter dose-response model.

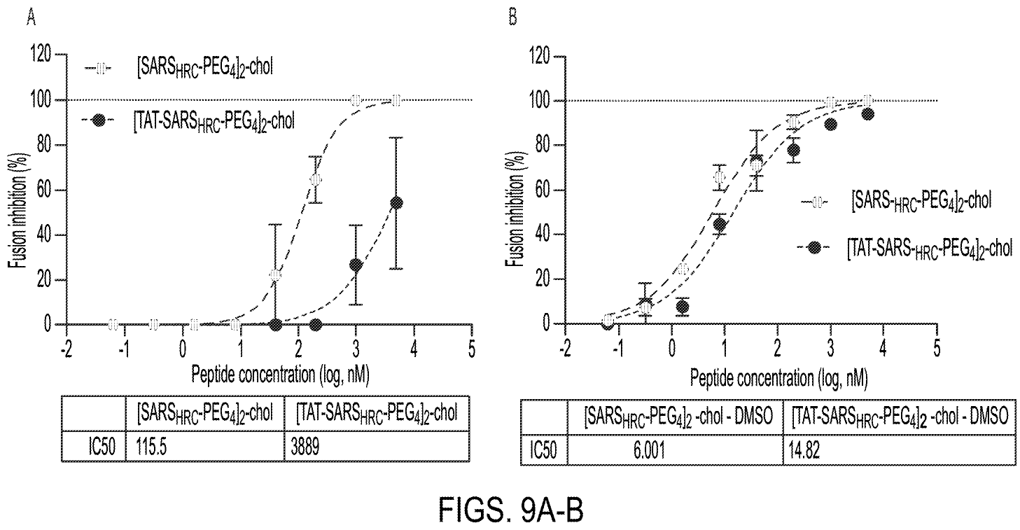

FIGS. 9A-B: Addition of cell penetrating peptide sequence does not increase the antiviral activity of [SARS_{HRC}-PEG₄]₂-chol. (A) TAT-SARS comparison-peptide efficacy comparison in VeroE6 cells. (B) TAT-SARS comparison-peptide efficacy comparison in VeroE6-TMPRSS2 cells. In both panels, the percentage inhibition of infection is shown on VeroE6 and VeroE6-TMPRSS2 cells with increasing concentrations of [SARSHRC-PEG4]2-chol (light blue lines) and [TAT-SARSHRC-PEG4]2-chol (dark blue lines).

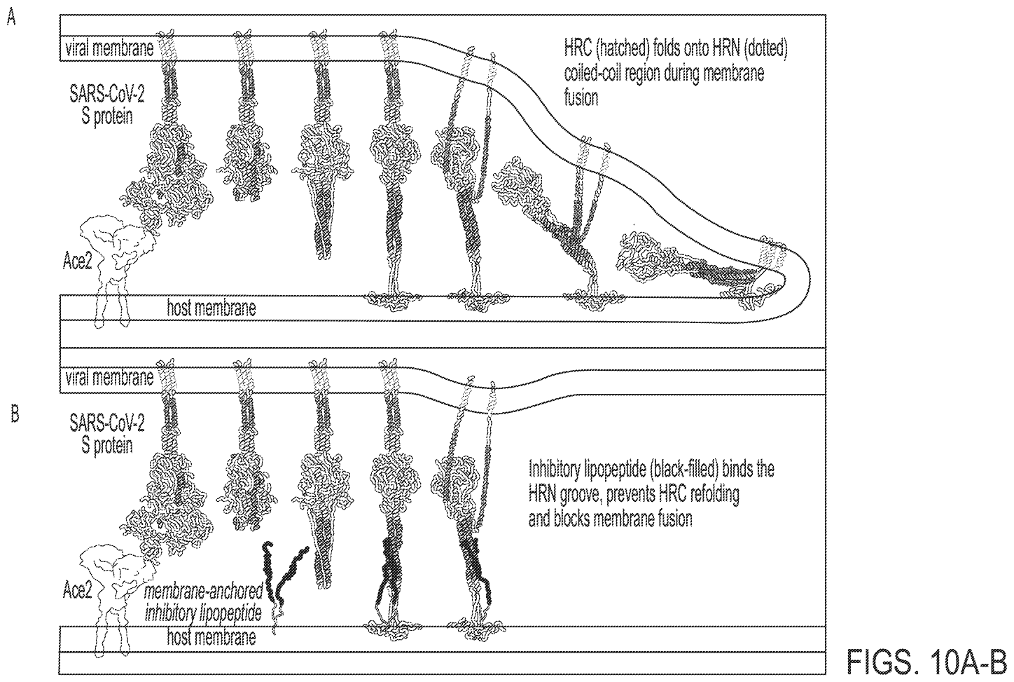

FIGS. 10A-B: Models for mechanism of virus-host cell membrane fusion. (A) Proposed model of interactions between S on the viral envelope and Ace2 on the host cell membrane leading to membrane fusion. (B) Proposed anchoring of the dimeric lipopeptide in the host cell membrane and interactions with the viral S protein to inhibit S-mediated fusion.

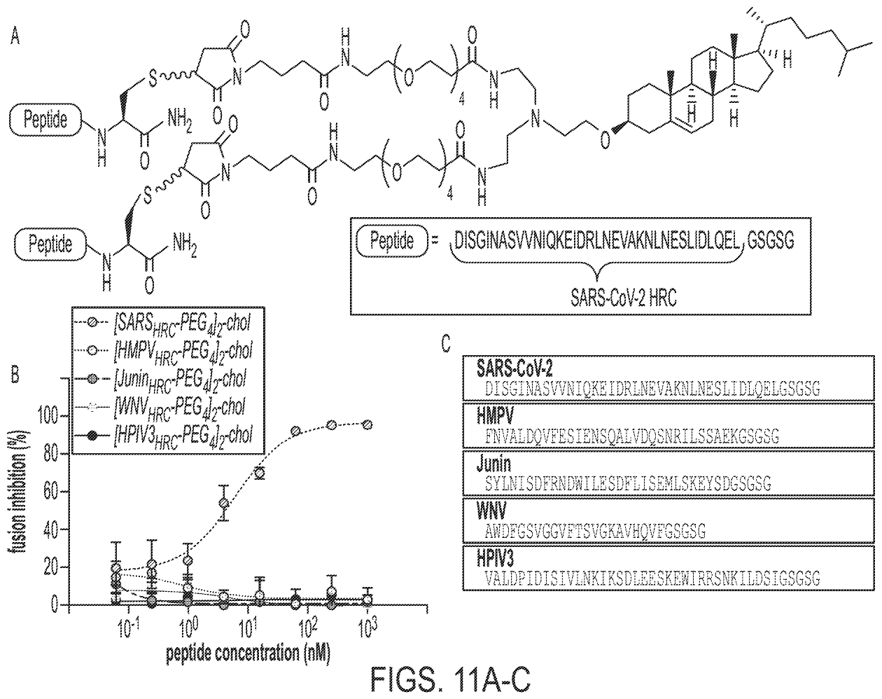

FIGS. 11A-C: Design and specificity of SARS-COV-2 inhibition by [SARS_{HRC}-PEG₄]₂-chol. (A) Chemical structure of [SARS_{HRC}-PEG₄]₂-chol. (B) [SARS_{HRC}-PEG₄]₂-chol proved specific, as lipopeptides based on the HRC domains from several other human pathogens did not inhibit S-mediated fusion at any concentration tested (human metapneumovirus=HMPV; West Nile virus=WNV; human parainfluenza virus type 3=HPIV3). Percent fusion inhibition was calculated as the ratio of relative luminescence units in the presence of a specific concentration of inhibitor and the relative luminescence units in the absence of inhibitor and corrected for background luminescence. Data are means±standard deviation (SD). (C) Sequences of respective peptides evaluated in FIG. 11B.

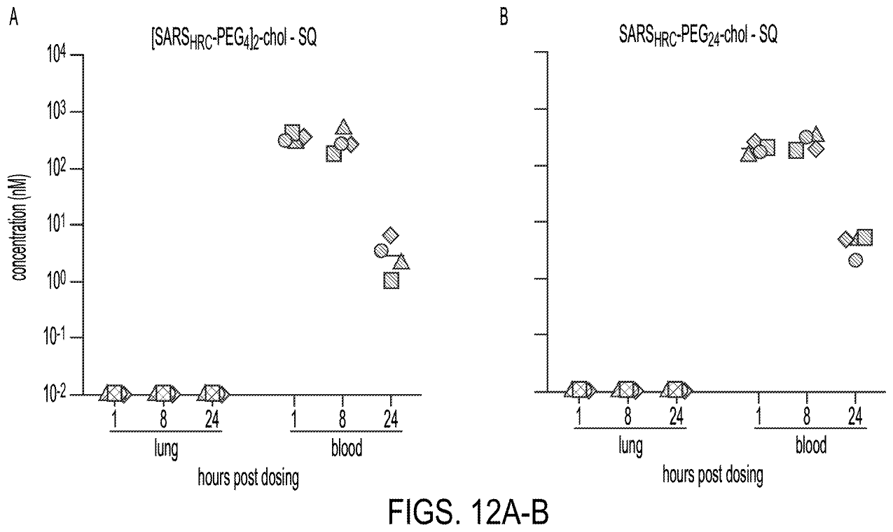

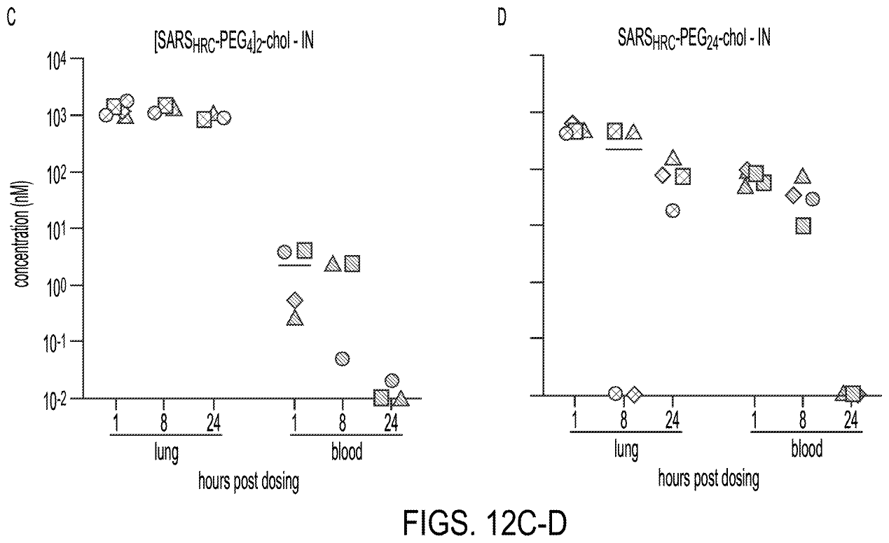

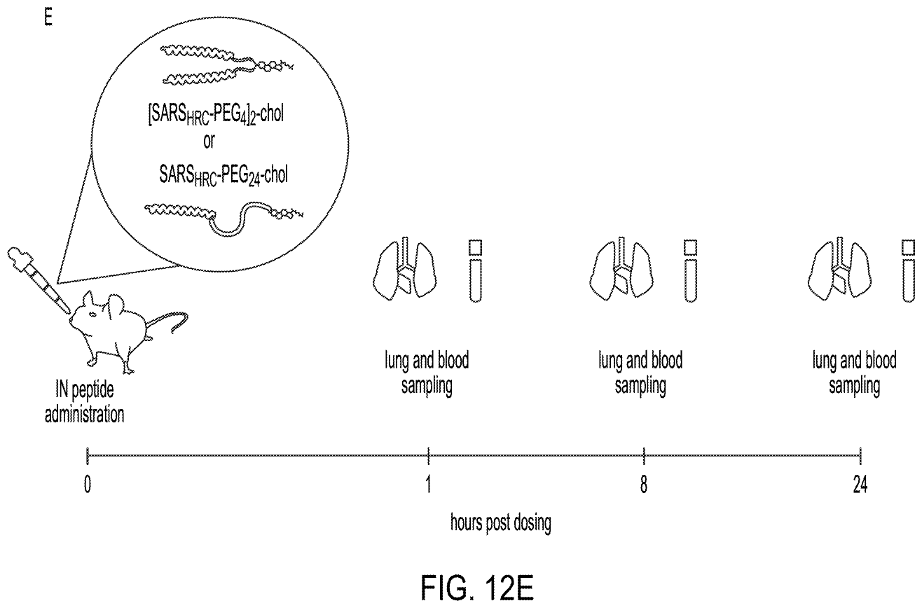

FIGS. 12A-E: In vivo biodistribution assessment. (A, B) Mice were subcutaneously (SQ) injected with [SARS_{HRC}-PEG₄]₂-chol and SARS_{HRC}-PEG₂₄; lungs and blood were harvested at 1, 8 and 24 hours post dosing. The concentration of lipopeptides (y-axis) was measured by ELISA in lung homogenates and plasma samples (peptide-treated n=3 or 4). n=1 mock-treated mouse was included as negative control. The experiment was conducted in triplicate for each ELISA point. Median is indicated by horizontal bar. (C, D) Similar experiment performed after intranasal administration. (E) Experimental design of biodistribution experiment in hACE2 transgenic mice. Mice were intranasally (IN) inoculated with [SARS_{HRC}-PEG₄]2-chol and SARS_{HRC}-PEG₂₄ and lungs and blood were harvested at 1, 8 and 24 hours post dosing.

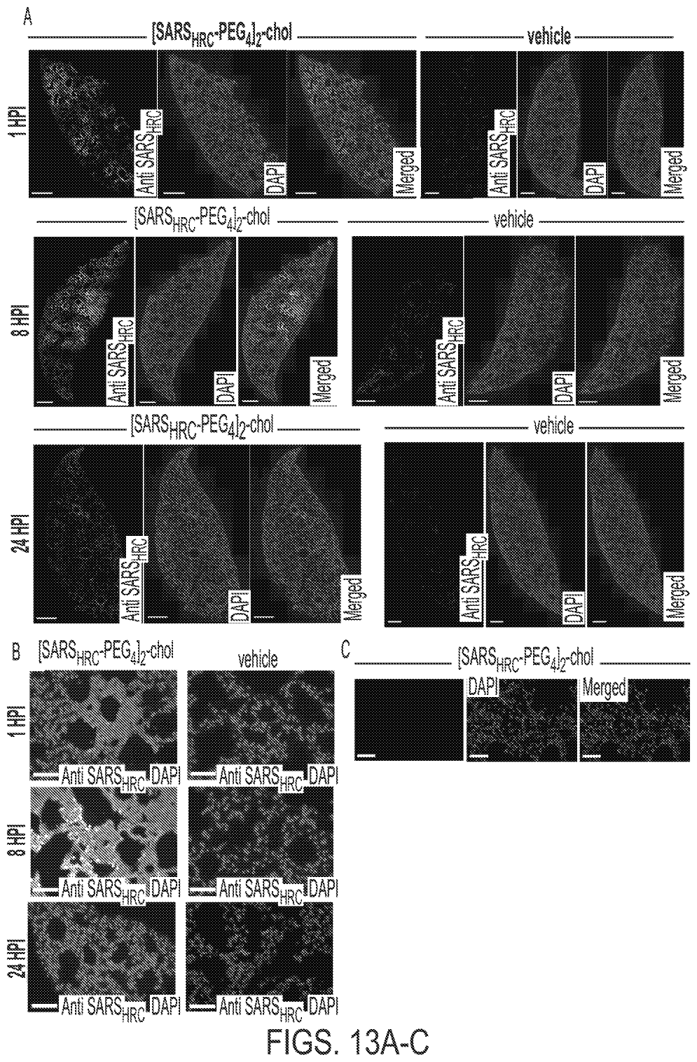

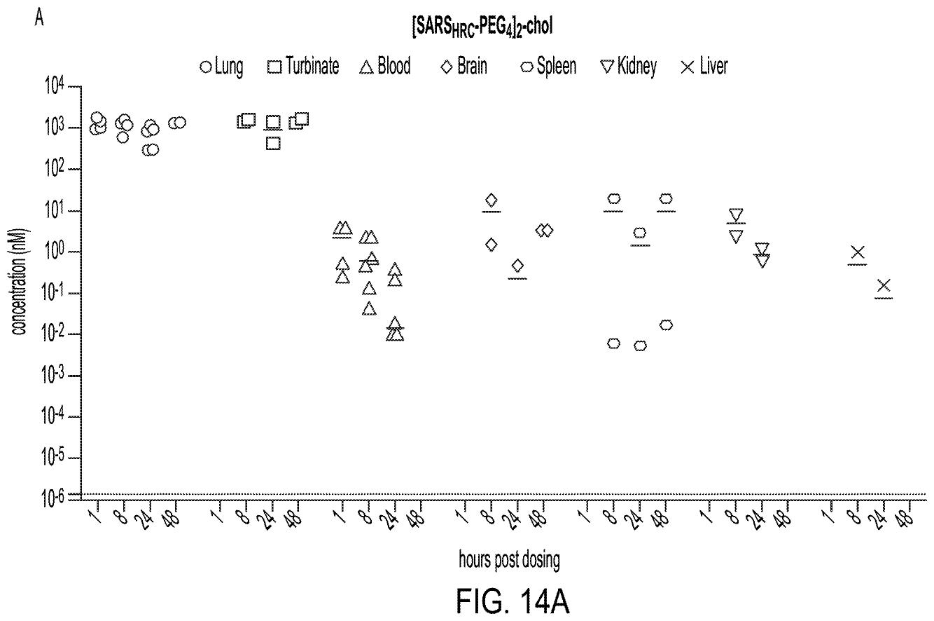

FIG. 13: Lung distribution of [SARS_{HRC}-PEG₄]2-chol-treated (or vehicle-treated) mice were stained with anti-SARS-HRC antibody (red) and nuclei were counterstained with DAPI (blue). The images confirmed broad distribution of [SARS_{HRC}-PEG₄]₂-chol in lung sections of treated animals compared to those treated with vehicle, at 1, 8, 24 hours post-inoculation (HPI). (A) lung tile scans, scale bar=500 μm (B), 40× images, scale bar=50 μm (C) Antibody specificity test. Lung section of a [SARS_{HRC}-PEG₄]2-chol-treated mouse stained only with secondaries antibody did not show any cross-reactivity signals.

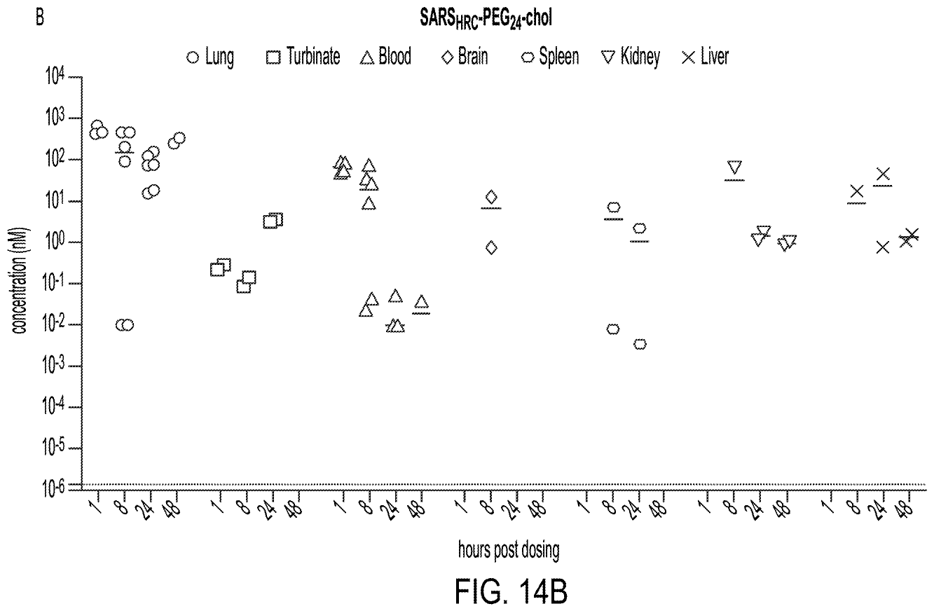

FIGS. 14A-B: In vivo biodistribution assessment. Mice were intranasal injected (IN) with (A) [SARS_{HRC}PEG₄]₂-chol or (B) SARS_{HRC}-PEG24. Organs and blood were harvested at 1, 8, 24 and 48 hours post dosing (n=2-6 mice). The concentration of lipopeptides (y-axis) was measured by ELISA in lung homogenates, turbinate, plasma, brain, spleen, kidney and liver samples. Median is indicated by horizontal bar and the limit of detection is indicated by dotted line. As can be seen from the figure, biodistribution of the dimer lipid-peptide fusion inhibitor in the turbinate and in the lungs was comparable, with equivalent concentrations maintained at 1, 8, 24 and 48 hours after intranasal administration. The biodistribution of the monomer lipid-peptide fusion inhibitor was strikingly different to the dimer, with lower levels of the monomer seen in the turbinate than the lungs at 1, 8, 24 and 48 hours after administration.

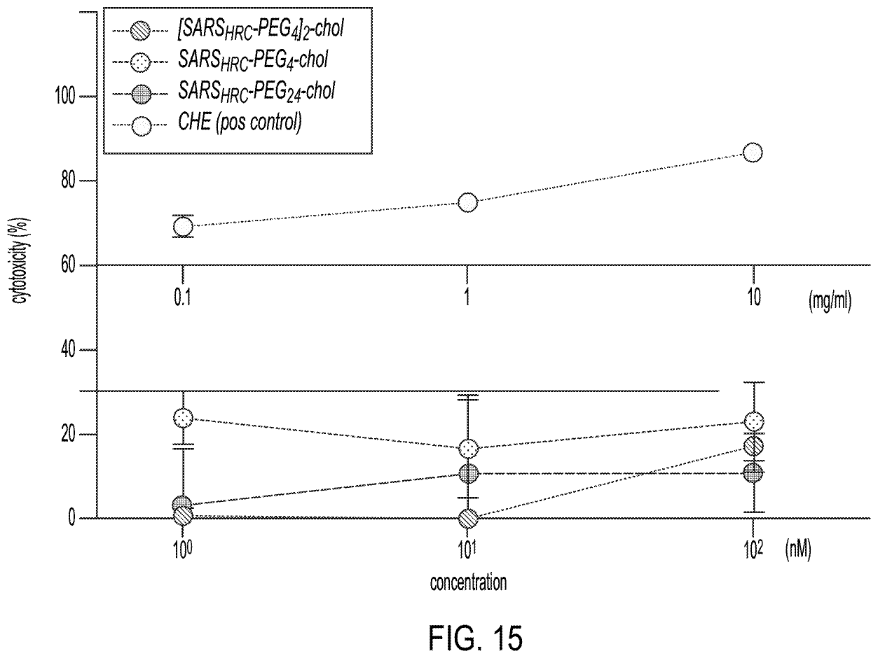

FIG. 15. Fox vivo cytotoxicity assessment. An MTT (3-[4,5-dimethylthiazole-2-yl]-2,5-diphenyltetrazolium bromide) assay was used to determine the toxicity of the [SARS_{HRC}-PEG₄]₂-chol, SARS_{HRC}-PEG₄-chol, and SARS_{HRC}-PEG₂₄-chol in human airway epithelial (HAE) cells. The toxicity observed for all lipopeptides was <30% even at the highest concentration tested (100 μM). Based on the lack of dose response, and the inherent variability of this ex vivo model, we consider 30% to be the variability range of this toxicity assay. Cycloheximide (CHE, at 0.1, 1 and 10 mg/ml on secondary x-axis, purple) was used as a positive control.

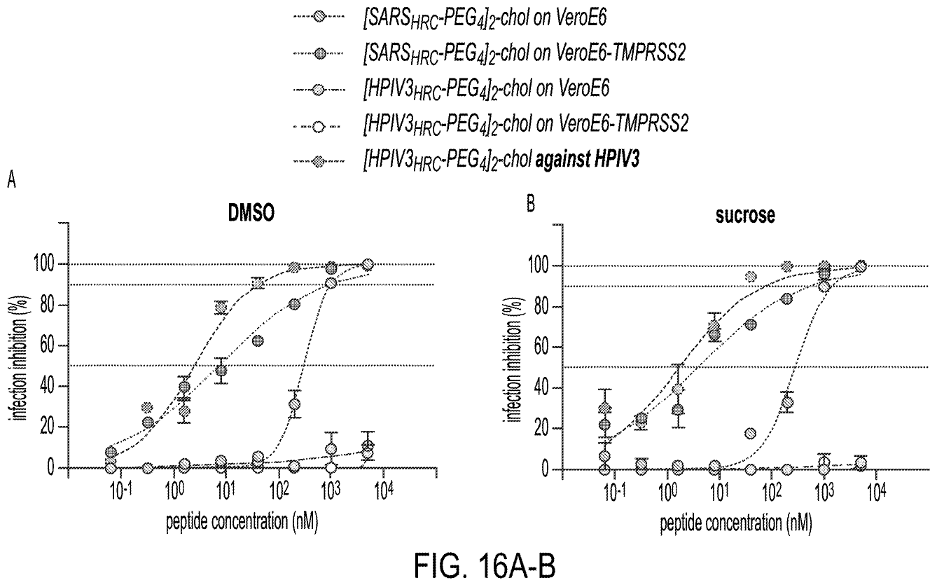

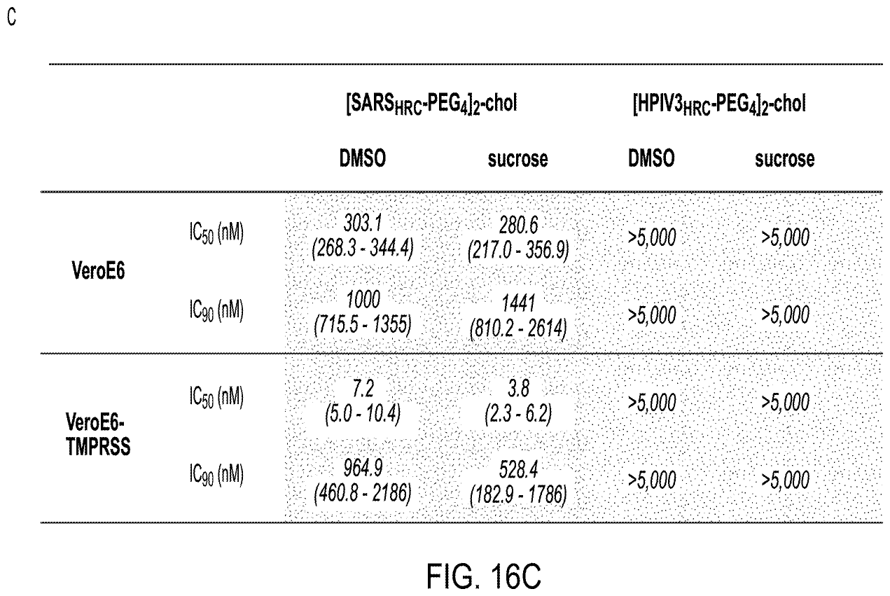

FIGS. 16A-C: Inhibition of infectious SARS-COV-2 entry by [SARS_{HRC}-PEG₄]₂-chol and [HPIV3_{HRC}-PEG₄]₂-chol peptides. (A, B) The percentage inhibition of infection is shown on VeroE6 and VeroE6-TMPRSS2 cells with increasing concentrations of [SARS_{HRC}-PEG₄]₂-chol (red lines) and [HPIV3_{HRC}-PEG₄]₂-chol (grey lines). DMSO-formulated (A) and sucrose-formulated stocks (B) were tested side-by-side. Mean±SEM of triplicates are shown, dotted lines show 50% and 90% inhibition. Additionally, the potency of [HPIV3_{HRC}-PEG₄]₂-chol was confirmed by inhibition of infectious HPIV3 entry (dotted green lines on Vero cells). (C) Inhibitory concentrations 50% and 90% of [SARS_{HRC}-PEG₄]₂-chol and [HPIV3_{HRC}-PEG₄]₂-chol against SARS-COV-2 were calculated by performing four parameter nonlinear regression with variable slope on the data shown in A and B. 95% confidence intervals are shown between brackets.

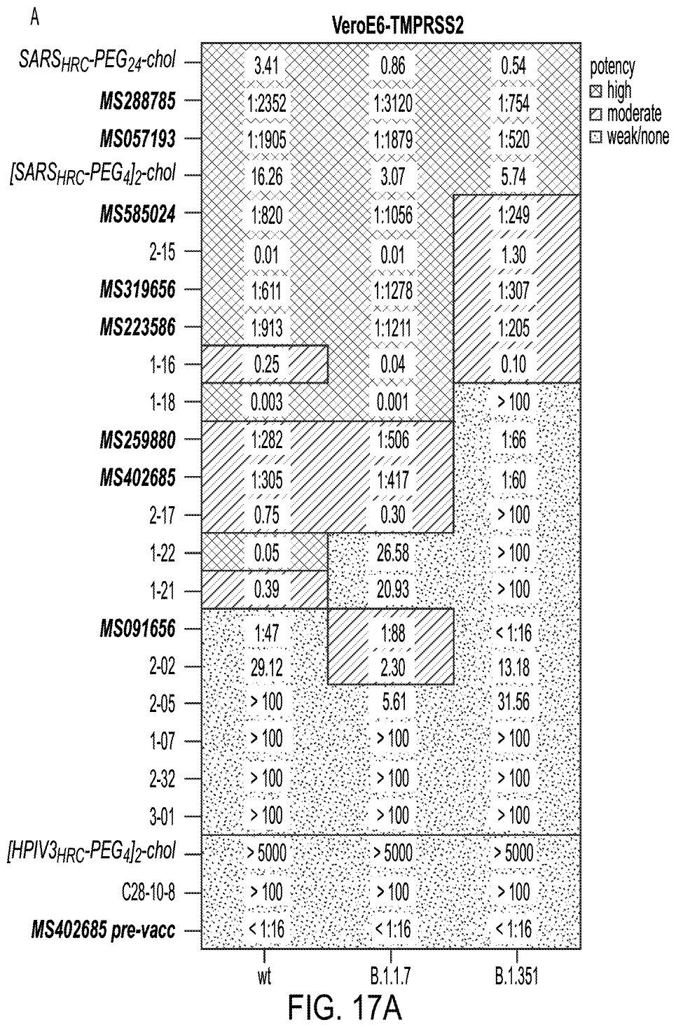

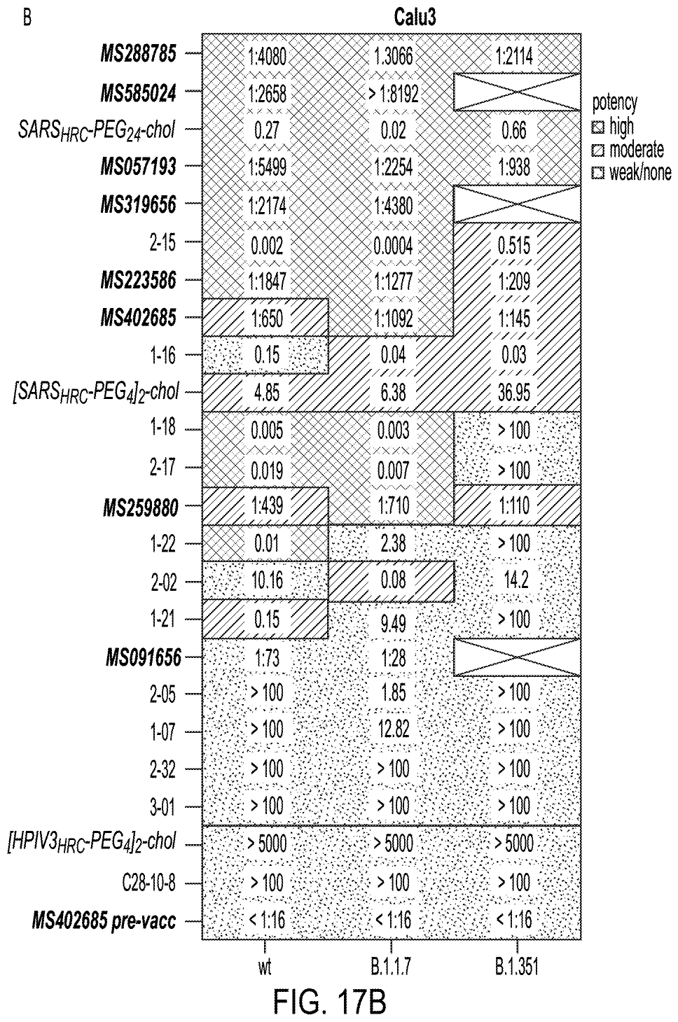

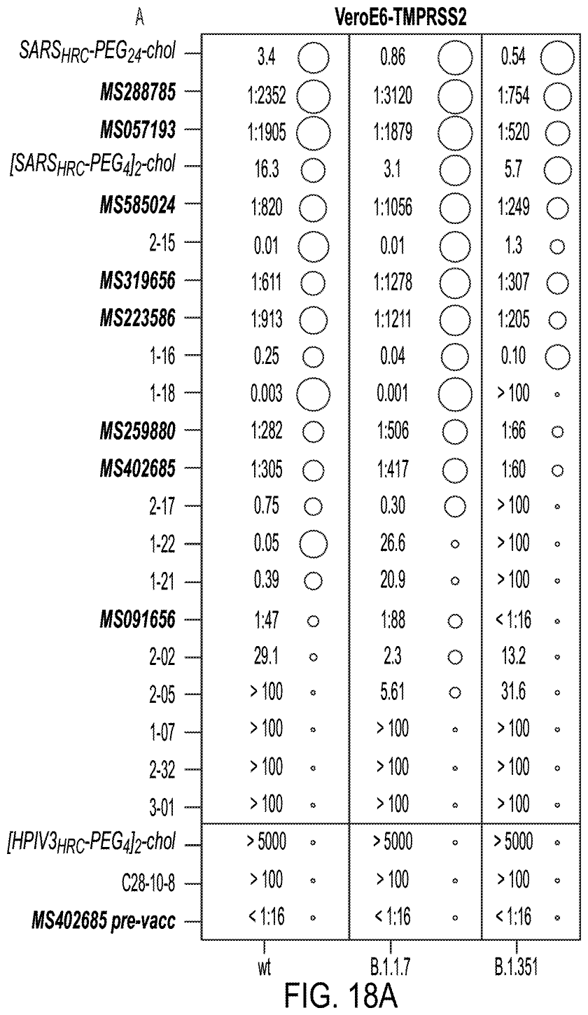

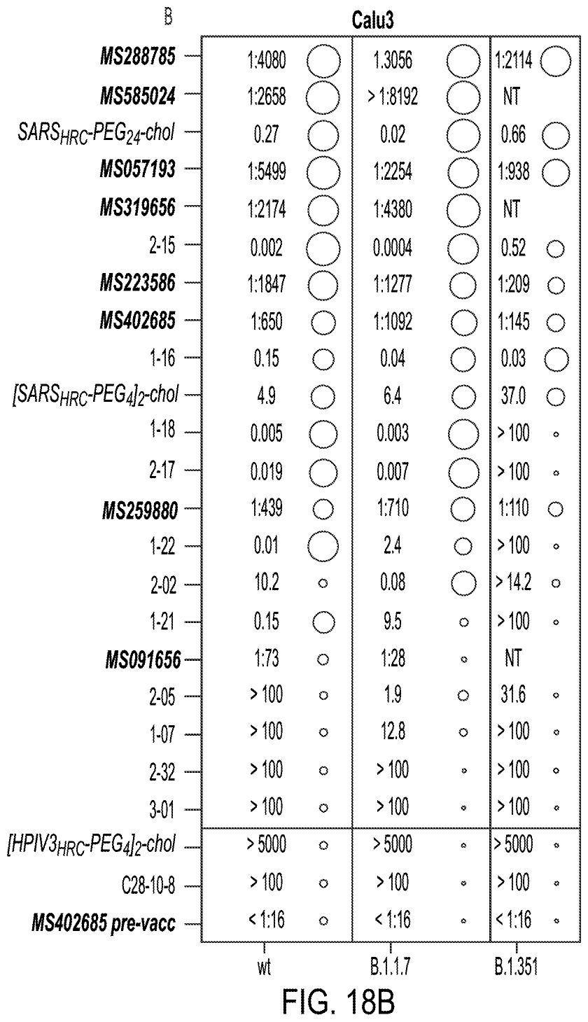

FIGS. 17A-B: Potency of inhibitory lipopeptides (FIPs), monoclonal antibodies (mAbs) or post-vaccination sera against entry of wt SARS-COV-2 and variants of concern (VOC). In an eight-hour infectious virus entry assay the efficacy of two FIPs, eleven mAbs and eight post-vaccination sera was tested in VeroE6-TMPRSS2 cells (A) and Calu3 cells (B). IC50 values were calculated using a four-parameter dose-response model, log-transformed into a range of 0-9 (reflecting the dilution series) and each inhibitor was ranked within its class (FIP, mAb, serum) into relative different potencies. Inhibitors are ordered based on potency and IC50 values are shown for the combination of each inhibitor and virus. IC50 values for FIPs (orange) are shown in nanomolar (nM), for mAbs (black) in ug/ml and for post-vaccination sera (purple) as dilution. For each class one negative control was included, which is shown below the line.

FIGS. 18A-B: Potency of inhibitory liopeptides (FIPs), monoclonal antibodies (mAbs) or post-vaccination sera against entry of wt SARS-COV-2 and variants of concern (VOC). In an eight-hour infectious virus entry assay the efficacy of two FIPs, eleven mAbs and eight post-vaccination sera was tested in VeroE6-TMPRSS2 cells (A) and Calu3 cells (B). IC50 values were calculated using a four-parameter dose-response model; symbol sizes represent relative response ranks. Briefly, the log-transformed response range (strongest to weakest response) was calculated per sample type (FIP, mAb, serum) and per cell type (VeroE6-TMPRSS2 or Calu3). The range was subdivided in ten ranks with equivalent distances, and each sample was assigned one of these ranks. IC50 values for FIPs (orange) are shown in nanomolar (nM), for mAbs (black) in ug/ml and for post-vaccination sera (purple) as dilution. For each class one negative control was included, which is shown below the line.

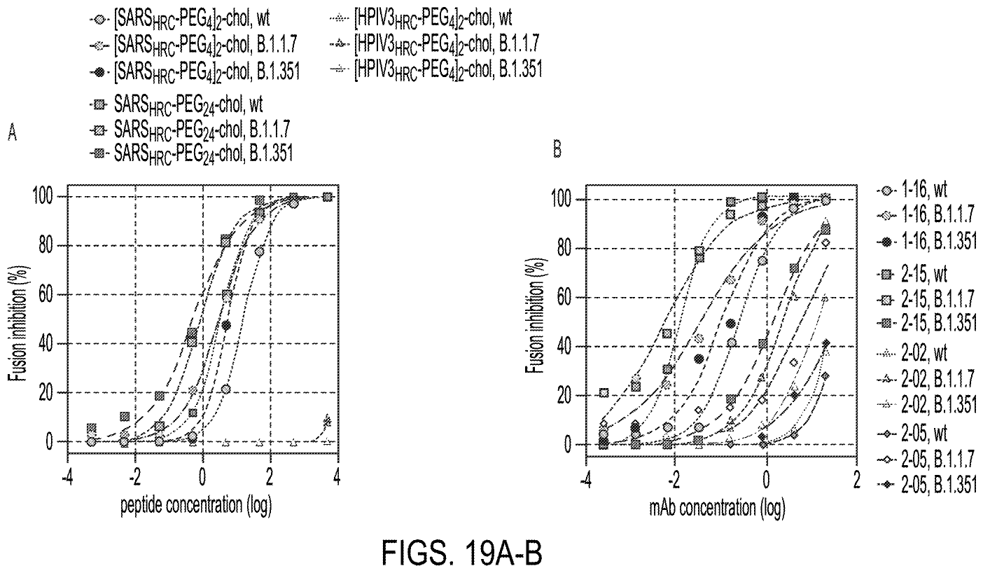

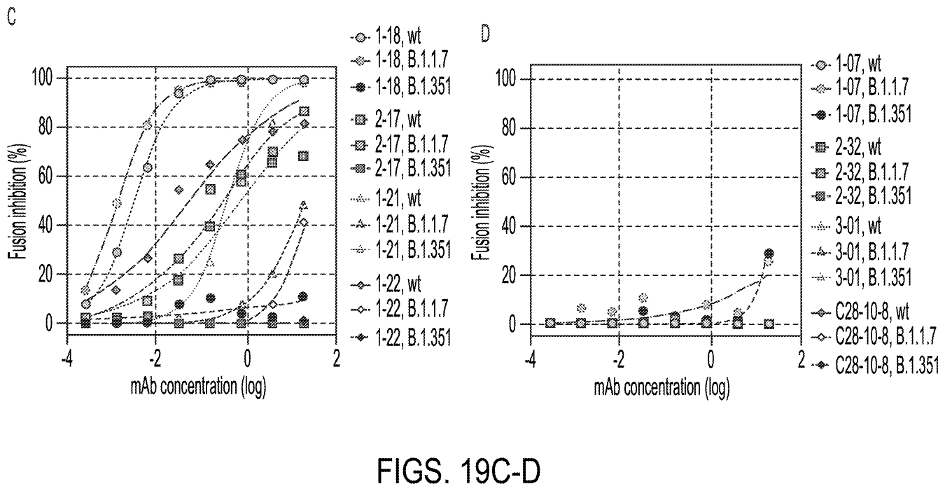

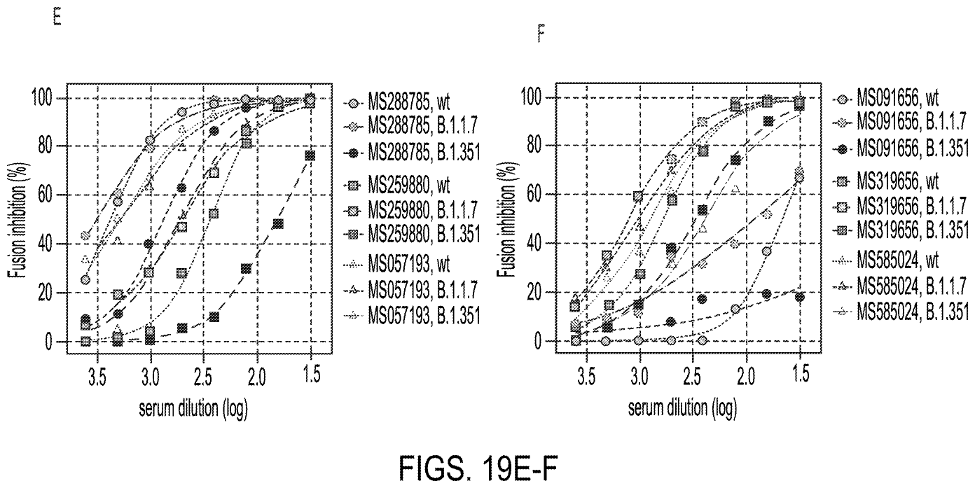

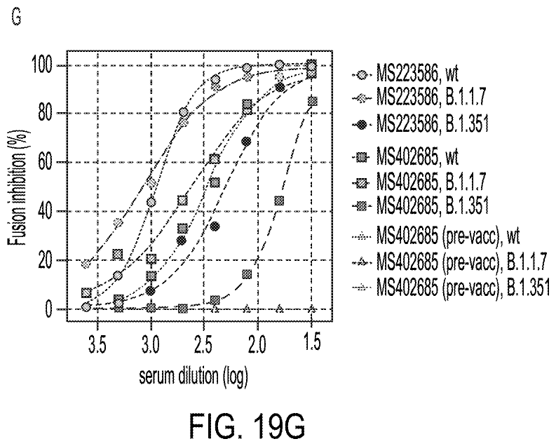

FIGS. 19A-G: Inhibition of wt SARS-COV-2 and VOC entry by fusion inhibitory peptides (FIPs), monoclonal antibodies (mAbs) or post-vaccination sera. In an eight-hour infectious virus entry assay the efficacy of two FIPs, eleven mAbs and eight post-vaccination sera was tested. Percentage entry inhibition in VeroE6-TMPRSS2 cells is shown for increasing concentrations of FIPs (A), mAbs (B-D) or increasing dilutions of post-vaccination sera (E-G). Red lines show wt SARS-COV-2, green lines show the alpha (B.1.1.7) and blue lines show the beta (B. 1.351) variant. All FIPs, mAbs and sera were tested side-by-side in triplicate and mean is plotted; curves represent a four-parameter dose-response model. A) FIPs were tested in a 10-fold dilution series from 0.0005 nM to 5000 nM. [HPIV3_{HRC}-PEG₄]₂-chol (HPIV-3-specific lipopeptide) was used as negative control. (B-D) mAbs were tested in a 5-fold dilution series from 0.0003 ug/ml to 20 ug/ml. MAb C28-10-8 (measles virus-specific monoclonal) was used as negative control. MAbs were classified according to activity to different VOC (B) active against all three tested viruses, (C) active against wt SARS-COV-2 and alpha (B.1.1.7), (D) not reactive). (E-G) Sera were tested in a two-fold dilution series ranging from 1:32 to 1:4096. Sera were obtained three weeks post two vaccinations with the BNT162b2 mRNA vaccine. A matched pre-vaccination sample was used as negative control.

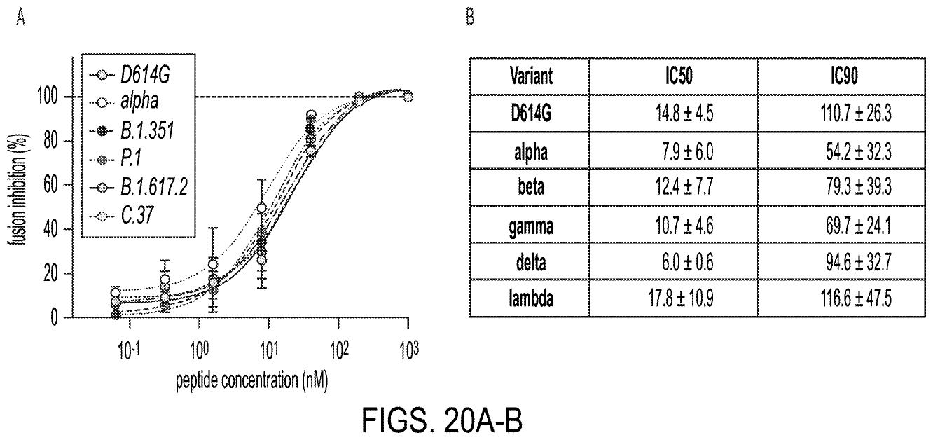

FIGS. 20A-B: Fusion inhibitory activity of [SARS_{HRC}-PEG₄]₂-chol peptide against emerging SARS-COV-2 S variants. (A) SARS-COV-2 glycoprotein and α-subunit of β-galactosidase with 293T cells transfected hACE2 receptor and ω-subunit of β-galactosidase was assessed by a β-galactosidase complementation assay in the presence of different dilutions of the peptide [SARS_{HRC}-PEG₄]₂-chol. Resulting luminescence from β-galactosidase was quantified using Tecan infinite M1000 pro. The values are means (±SEM) of results from three experiments. (B) Percent inhibition was calculated as the ratio of relative luminescence units in the presence of a specific concentration of inhibitor and the relative luminescence units in the absence of inhibitor and corrected for background luminescence as follows: percent inhibition=100×[1−(luminescence at X−background)/(luminescence in the absence of inhibitor−background)]. Data are means±standard errors (SE) (error bars) from three separate experiments with the curve representing a three-parameter dose-response model.

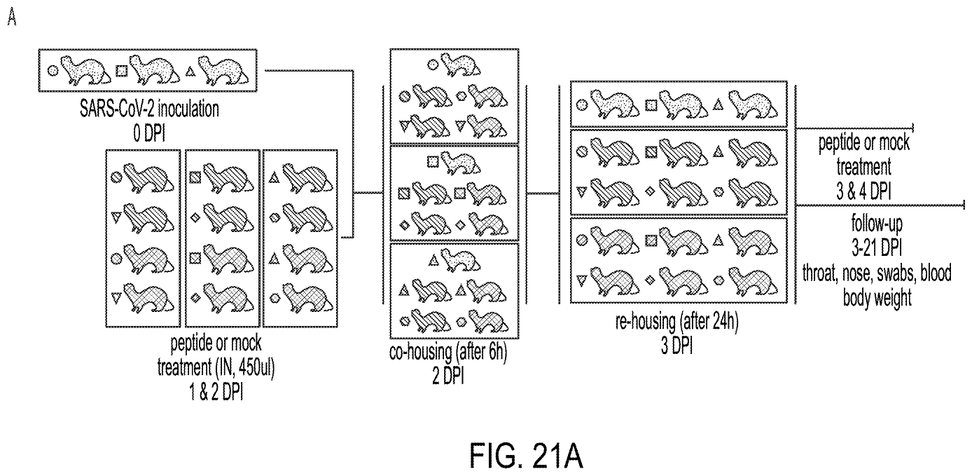

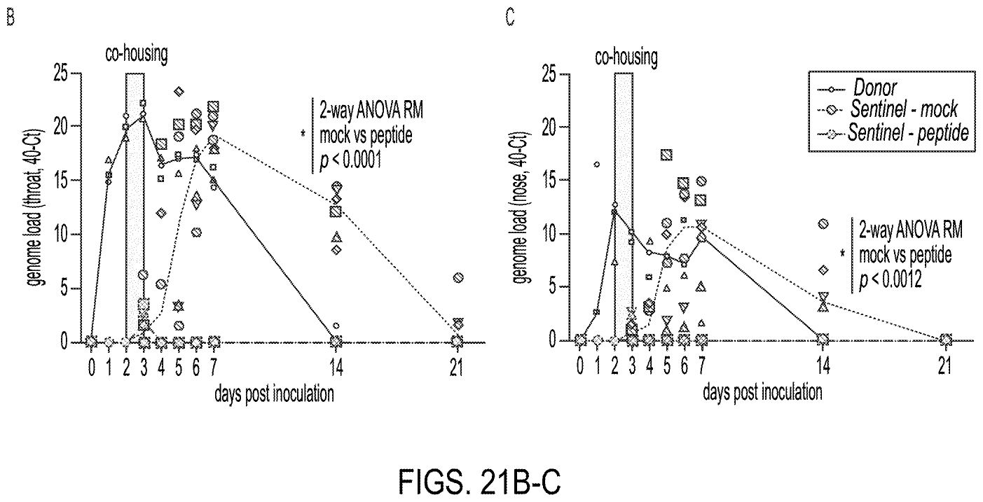

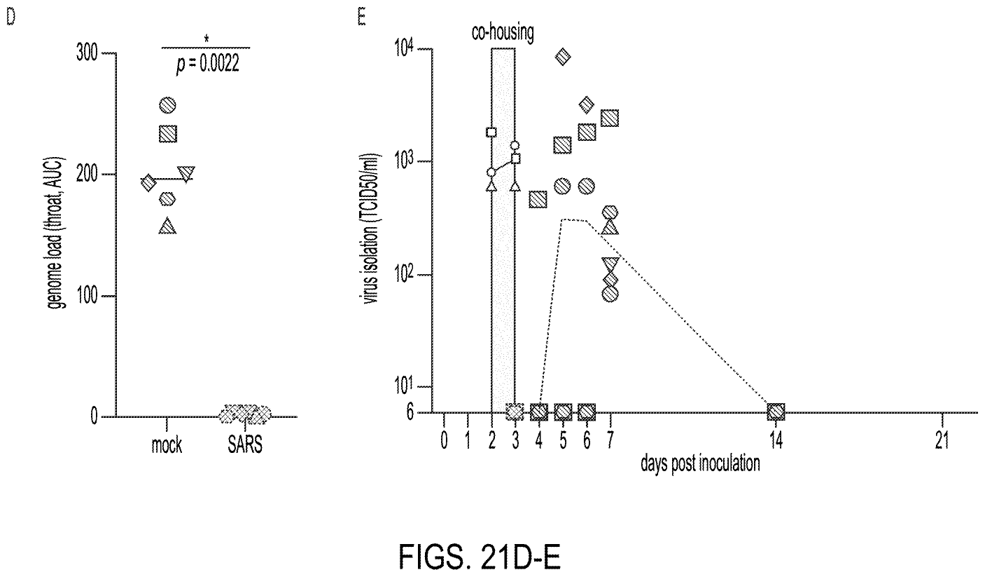

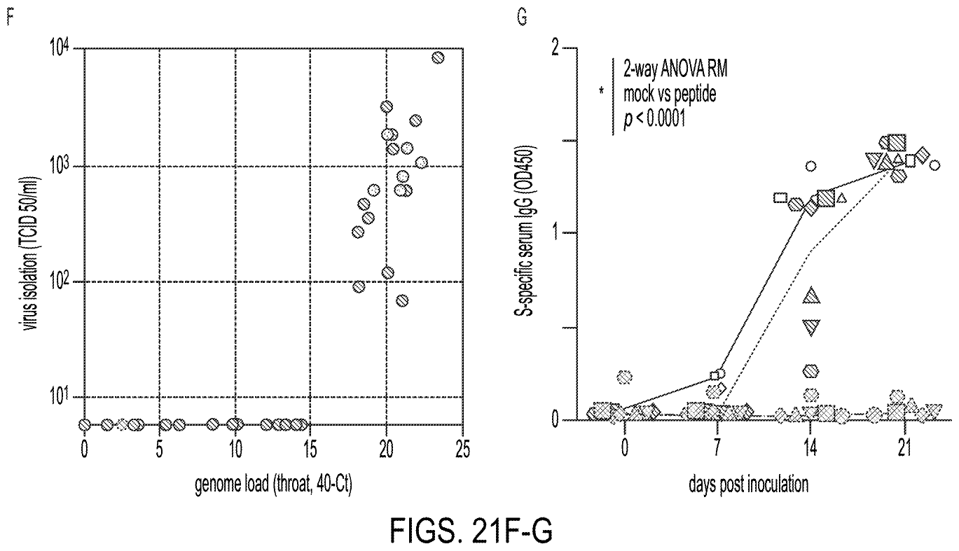

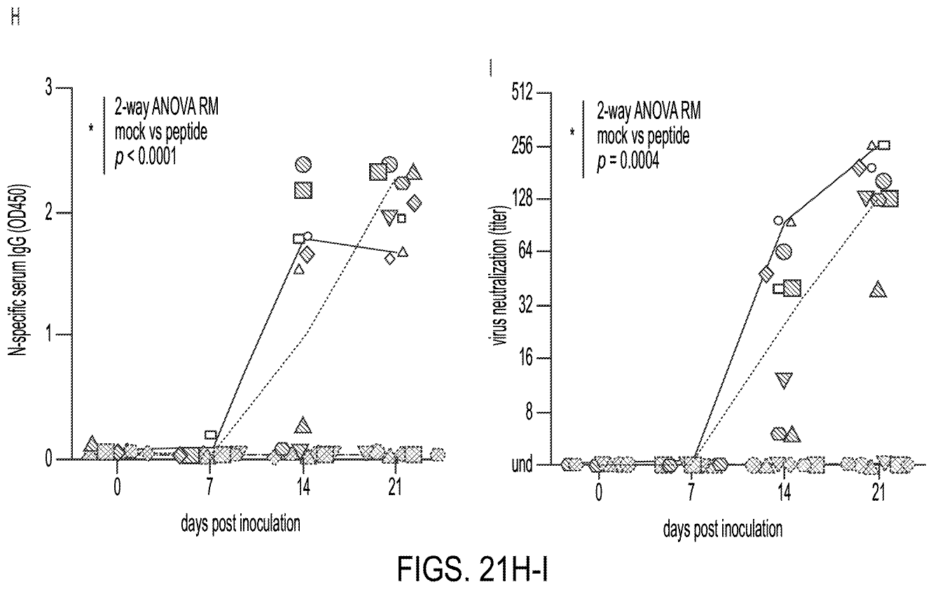

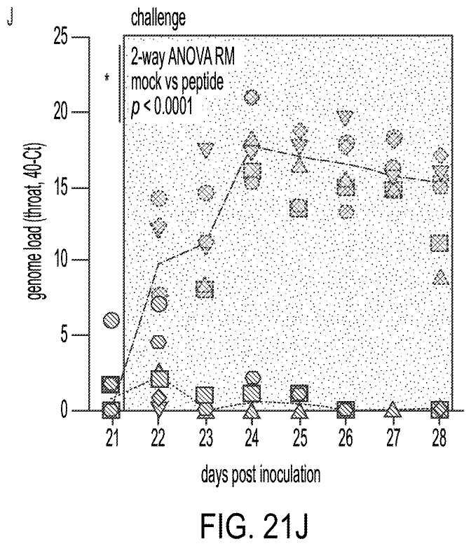

FIGS. 21A-J: [SARS_{HRC}-PEG₄]₂-chol prevents SARS-COV-2 transmission in vivo. (A) Experimental design. (B, C) Viral loads detected in throat (B) and nose (C) swabs by RT-qPCR. (D) Comparison of the area under the curve (AUC) from genome loads reported in B for mock- and peptide-treated sentinels. (E) Viral loads detected in throat swabs by live virus isolation on VeroE6. (F) Correlation between viral loads in the throat as detected via RT-qPCR and live virus isolation. Presence of anti-S(G) or anti-N(H) antibodies was determined by IgG ELISA assay. Presence of neutralizing antibodies was determined in a live virus neutralization assay (I) Virus neutralizing antibodies are displayed as the endpoint serum dilution factor that blocks SARS-COV-2 replication. (J) Direct inoculation of peptide-treated or mock-treated animals with SARS-COV-2 led to productive infection in only the previously peptide-treated animals, in the absence of S-specific, N-specific and neutralizing antibodies. Donor animals shown in grey, mock-treated animals in red, peptide-treated animals in green. Symbols correspond to individual animals (defined in A) and are consistent throughout figures. Line graphs in panels B, C, E and H-J connect the median of individual animals per time point. Mock- and peptide-treated groups were compared via 2-way ANOVA repeated measures (panels B, C, H-J) or Mann-Whitney test (panel D).

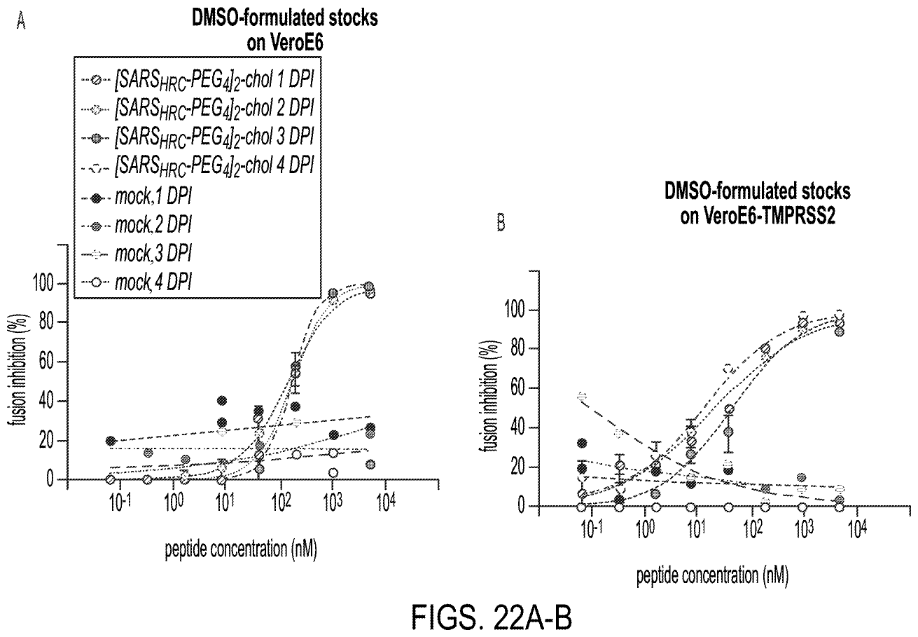

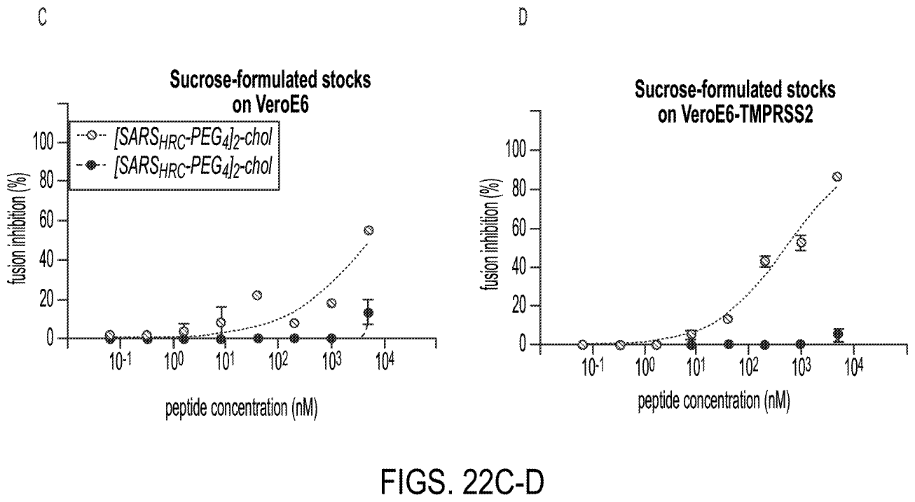

FIGS. 22A-D: In vitro potency of peptide stocks used in ferrets. (A, B) The potency of DMSO-formulated peptide dilutions used for intranasal inoculation of ferrets at 1-4 days post SARS-COV-2 inoculation (DPI, see FIG. 16A) was confirmed with a live virus infection assay. The percentage infection events are shown on (A) VeroE6 and (B) VeroE6-TMPRSS with increasing concentrations of [SARS_{HRC}-PEG₄]₂-chol (red) or mock (blue). The mock preparation was DI water with an equimolar amount of DMSO. Inhibitory concentrations 50% and 90% against SARS-COV-2 were calculated by performing four parameter nonlinear regression with variable slope, and were equivalent for all preparations. Data are means±standard error of the mean (SEM) from triplicates for peptide-dosing stocks, mock-dosing stocks were tested as single replicate. (C, D) The potency of sucrose-formulated peptide dilutions used for intranasal inoculation of ferrets at 1 day post inoculation (DPI, see FIG. 19a) was tested with a live virus infection assay. The percentage infection events is shown on (C) VeroE6 and (D) VeroE6-TMPRSS with increasing concentrations of [SARS_{HRC}-PEG₄]₂-chol (red) or [HPIV3_{HRC}-PEG₄]₂-chol (blue). Inhibitory concentrations 50% and 90% against SARS-COV-2 were calculated by performing four parameter nonlinear regression with variable slope. Data are means±standard error of the mean (SEM) from triplicates. The 10-100 fold higher IC₅₀ and IC₉₀ obtained with sucrose-formulated lipopeptide generated at 10 mg scale as compared to DMSO-formulated lipopeptide (compare panels C/D with panels A/B) was subsequently confirmed using in vitro fusion assays (data not shown).

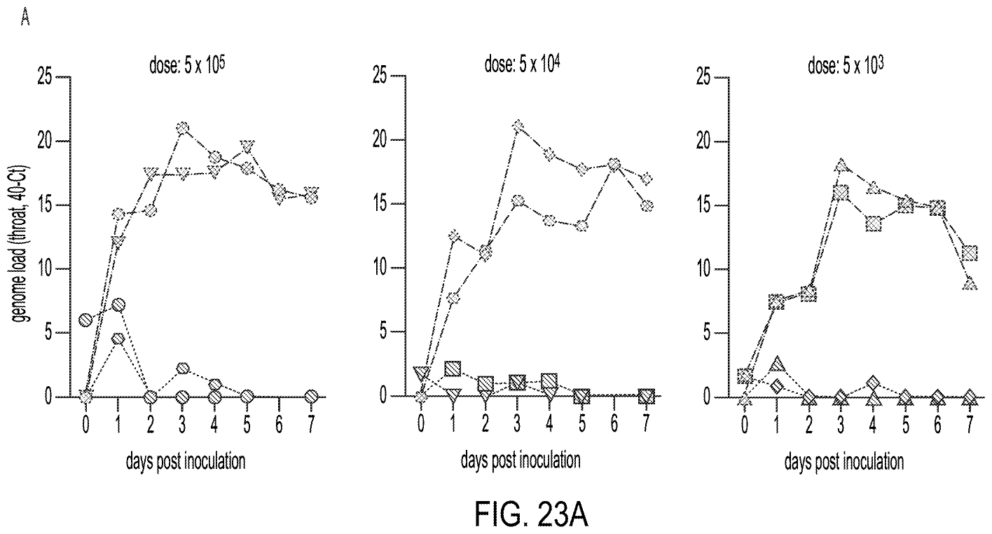

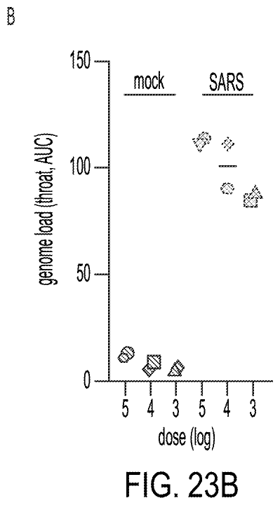

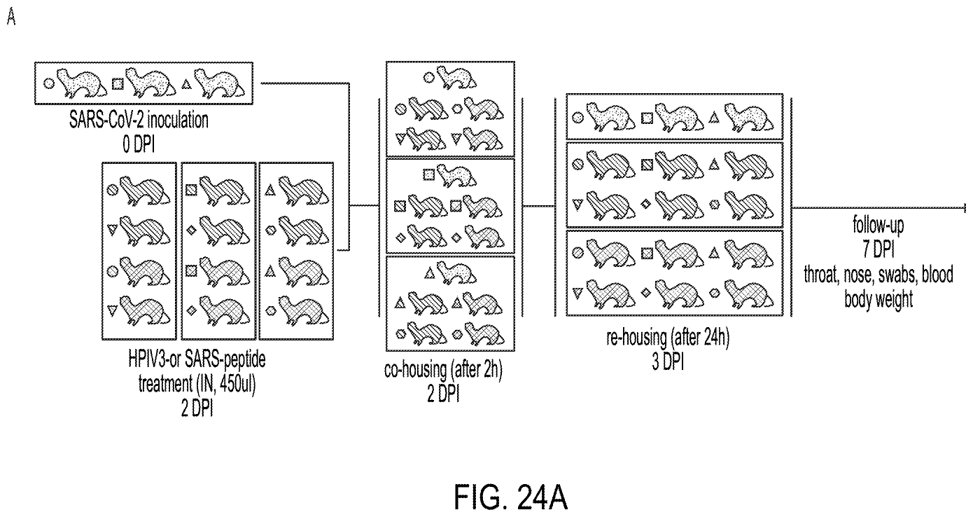

FIGS. 23A-B: Challenge infection of previously peptide-treated and mock-treated animals with SARS-COV-2. To confirm absence of antibodies as an accurate measure of sterile protection, previously mock-treated or [SARS_{HRC}-PEG₄]₂-chol-treated ferrets were challenged with infectious SARS-COV-2 (see FIG. 16j). Ferrets were re-housed in pairs of the same treatment schedule into six isolators and challenged with 5×10⁵, 5×10⁴ or 5×10³ TCID₅₀/ml (in 450 μl) of SARS-COV-2. For each dose, two mock-treated and two peptide-treated ferrets were inoculated intranasally. Viral loads in throat swabs were determined on a daily basis by RT-qPCR up to 7 days post inoculation, when the experiment was ended (A). Line graphs represent individual animals, symbols correspond to symbols as described in FIG. 16A: red is mock-treated and green is peptide-treated. (B) Area under the curves (AUC) demonstrate that the total genome load slightly decreases corresponding to the challenge dose. Since only two animals were included per group, statistics were not performed.

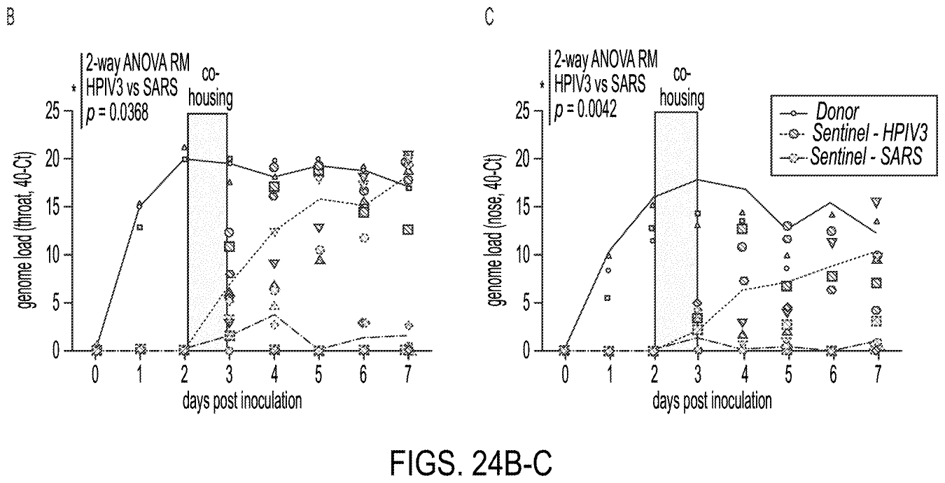

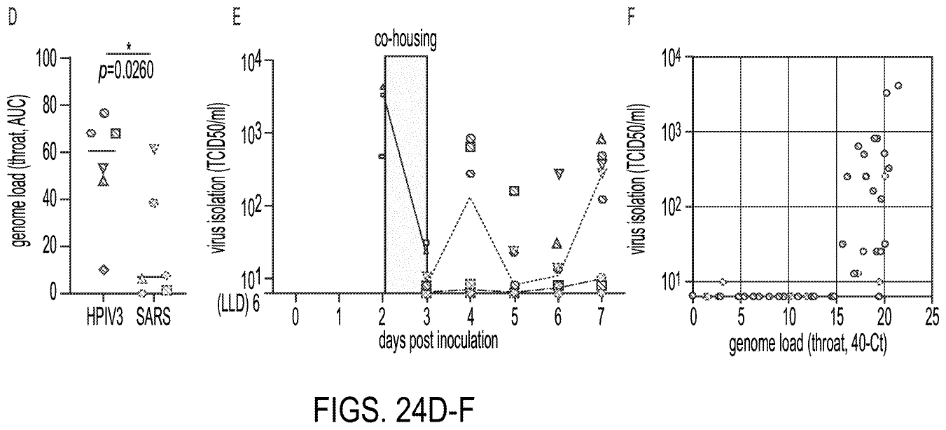

FIGS. 24A-F: A single dose of [SARS_{HRC}-PEG₄]₂-chol provides protection against SARS-COV-2 transmission in vivo. (A) We assessed the potential for a single administration of sucrose-formulated lipopeptide two hours before co-housing to prevent or delay infection, using an HPIV3-specific peptide as mock control. (B,C) Viral loads detected in throat (B) and nose (C) swabs by RT-qPCR. (D) Comparison of the area under the curve (AUC) from genome loads reported in B for [HPIV3_{HRC}-PEG₄]₂-chol-treated and [SARS_{HRC}-PEG₄]₂-chol-treated sentinels. (E) Viral loads detected in throat swabs by live virus isolation on VeroE6. (F) Correlation between viral loads in throat determined by RT-qPCR or infectious virus isolation. Infectious virus could only by isolated in throat swabs with a 40-Ct>15. Donor animals shown in grey, mock-treated animals in red, peptide-treated animals in green. Symbols correspond to individual animals (defined in A) and are consistent throughout figures (except in F). Line graphs in panel B, C and E are the median of individual animals per time point. Mock- and peptide-treated groups were compared via 2-way ANOVA repeated measures (panel B and C) or Mann-Whitney test (panel D). (F). Collectively, the SARS-COV-2 lipopeptide provided a significant level of protection as compared to the HPIV3 lipopeptide control group, but protection was not absolute and two out of six SARS-CoV-2 peptide-treated animals experienced breakthrough infection. Back-titration of the lipopeptides used for dosing revealed that the sucrose-formulated [SARS_{HRC}-PEG₄]₂-chol lipopeptide had been administered at a substantially lower concentration than the experiment with DMSO-formulated lipopeptide (FIG. 17).

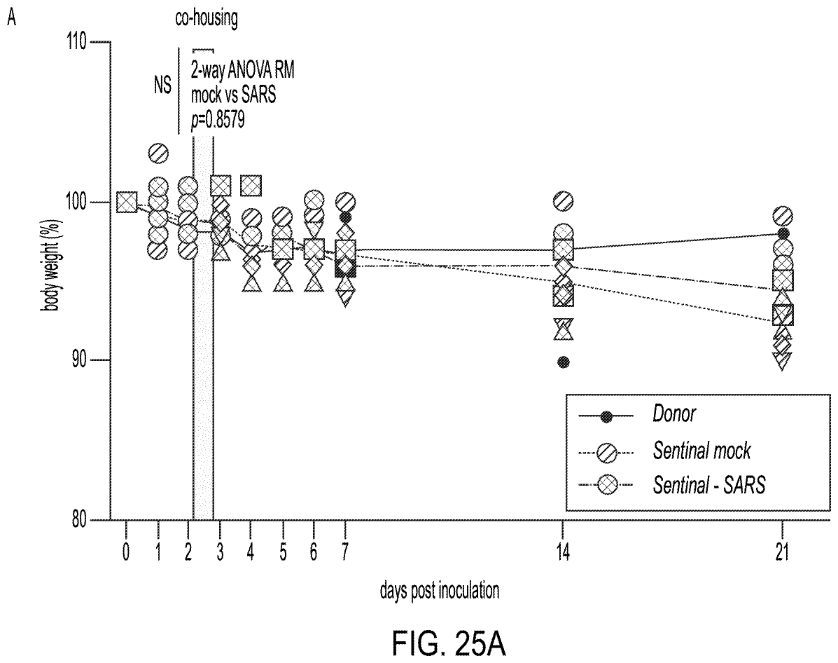

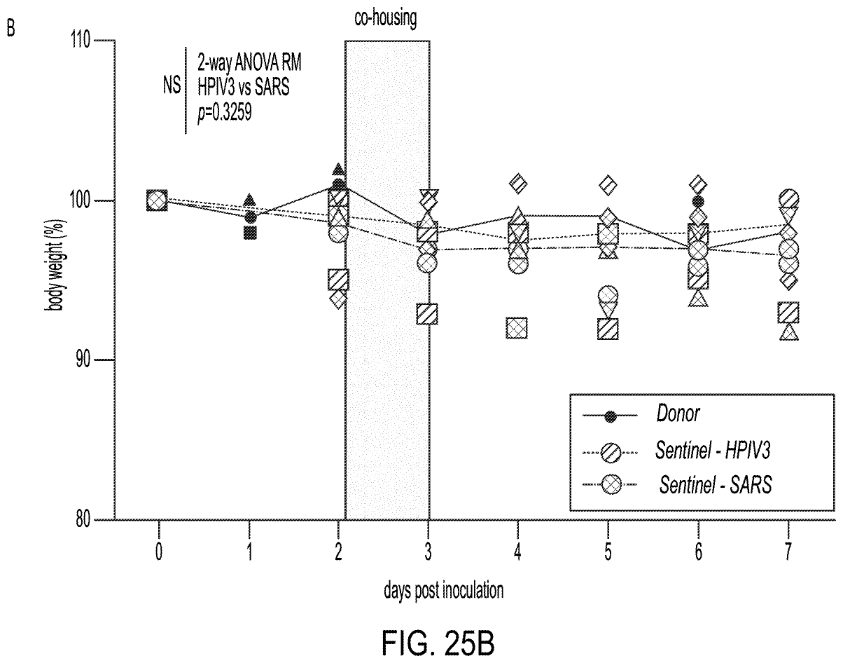

FIGS. 25A-B: Weight loss in control- and peptide-treated ferrets is not significantly different. Body weights of ferrets remained stable over time in both the experiment with DMSO-formulated peptides (A, corresponds to experiment described in FIG. 16A) and sucrose-formulated peptides (B, corresponds to experiment described in FIG. 19A). Donor animals are shown in grey, control-treated animals in red, [SARS_{HRC}-PEG₄]₂-chol-treated animals in green. Symbols correspond to individual animals as described in FIG. 16A and FIG. 19A. Line graphs are the median of individual animals per time point. Groups were compared via 2-way ANOVA repeated measures, significant differences between donor, mock-treated, [SARS_{HRC}-PEG₄]₂-chol-treated and [HPIV3_{HRC}-PEG₄]₂-chol-treated ferrets were not observed (NS=non-significant).

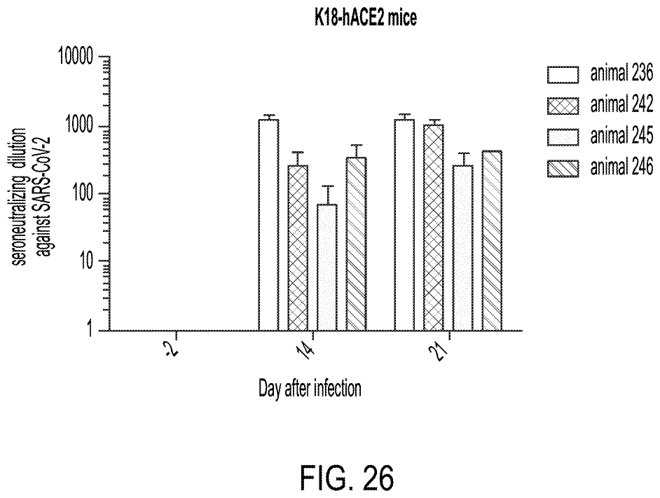

FIG. 26. In vivo efficacy of SARS peptides in transgenic mice expressing the human ACE2 receptor. SARS peptides and were administered to transgenic mice expressing the human ACE2 receptor, under the control of cytokeratin K18 (B6.Cg-Tg (K18-ACE2) 2Prlmn/J, Jackson) promoter intranasally, prior to infection with SARS-COV-2. Mice were pre-treated with peptide. The animals were challenged with virus at day 21 and all survived (data not shown). The surviving mice were assessed for the presence of neutralizing antibodies, and the titers are shown at day 14 and 21. The SARS peptides used include [SARS_{HRC}-PEG₄]₂-chol (for FIG. 21) and SARS_{HRC}-PEG₂₄-chol (data not shown).

### DETAILED DESCRIPTION OF THE INVENTION

The invention covers lipid-peptide molecules for the prevention and treatment of COVID-19. The invention uses designed peptides that block SARS-COV-2 entry into cells and will likely prevent and/or abrogate infection in vivo and prevent transmission. The designed lipid-peptide molecules are highly effective at inhibiting live SARS-COV-2 (COVID) virus infection in cultured cells and animal models.

Infection by coronaviruses, including the SARS-COV-2 (COVID) virus, requires membrane fusion between the viral envelope and the lung cell membrane. The fusion process is mediated by the virus's envelope glycoprotein, also called spike protein or S. The inventors engineered specific lipid-peptide constructs, that inhibit viral fusion and infection by binding to transitional stages of the spike protein, therefore preventing its function. Importantly, these antivirals can be given by the airway, by nasal drops or other method of nasal administration including powder, are not toxic, and have good half-life in the lungs. The fact that they can be given via the nose and inhalation makes them convenient and feasible for widespread use. Testing the lead antivirals in animal models will show utility for preventing and treating infection and preventing contagion from an infected animal to a healthy animal, including treatment as nasal drops or spray to prevent infection of healthcare workers.

In certain aspects, the invention provides a peptide; the C-terminal part of the peptide is “Gly-Ser-Gly-Ser-Cys,” and the N-terminal part of the peptide is selected from SEQ ID NO:1 and SEQ ID NO:2. In certain aspects, the invention provides a peptide; the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2.

In certain aspects, a SARS lipid-peptide fusion includes a lipid tag, a peptide where the C-terminal part of the peptide is “Gly-Ser-Gly-Ser-Cys,” and the N-terminal part of the peptide is selected from SEQ ID NO:1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2.

In some embodiments, the lipid tag is Cholesterol, Tocopherol, or Palmitate. In some embodiments, the lipid tag is Cholesterol.

In certain aspects, a SARS lipid-peptide fusion inhibitor includes a lipid tag, a spacer, a peptide where the C-terminal part of the peptide is “Gly-Ser-Gly-Ser-Cys,” and the N-terminal part of the peptide is selected from SEQ ID NO:1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO: 1 and SEQ ID NO:2.

In some embodiments, the spacer is a polyethylene glycol (PEG). In some embodiments, the spacer is PEG₄, PEG₁₁, or PEG₂₄. In some embodiments, the lipid tag is Cholesterol, Tocopherol, or Palmitate. In some embodiments, the lipid tag is Cholesterol.

In some embodiments, the SARS lipid-peptide fusion inhibitor has one peptide moiety, one spacer moiety, and one lipid tag. In some embodiments, the inhibitor has two peptide moieties, two spacer moieties, and one lipid tag. The terms “linker” and “spacer” are used interchangeably in the instant application.

In certain aspects, a pharmaceutical composition includes a peptide where the C-terminal part of the peptide is “Gly-Ser-Gly-Ser-Cys,” and the N-terminal part of the peptide is selected from SEQ ID NO: 1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2, and a pharmaceutically acceptable excipient.

In certain aspects, a pharmaceutical composition includes a peptide where the C-terminal part of the peptide is “Gly-Ser-Gly-Ser-Cys,” and the N-terminal part of the peptide is selected from SEQ ID NO:1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2, a lipid tag, and a pharmaceutically acceptable excipient.

In some embodiments, the lipid tag is Cholesterol, Tocopherol, or Palmitate.

In certain aspects, a pharmaceutical composition includes a peptide where the C-terminal part of the peptide is “Gly-Ser-Gly-Ser-Cys,” and the N-terminal part of the peptide is selected from SEQ ID NO: 1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2, a lipid tag, a spacer, and a pharmaceutically acceptable excipient.

In some embodiments, the spacer is a polyethylene glycol (PEG). In some embodiments, the spacer is PEG₄, PEG₁₁, or PEG₂₄. In some embodiments, the lipid tag is Cholesterol, Tocopherol, or Palmitate.

In some embodiments, the SARS lipid-peptide fusion inhibitor in the pharmaceutical composition has one peptide moiety, one spacer moiety, and one lipid tag. In some embodiments, the inhibitor has two peptide moieties, two spacer moieties, and one lipid tag.

In certain aspects, a SARS-COV-2 (COVID-19) antiviral composition includes a SARS-COV-2 (COVID-19) lipid-peptide fusion inhibitor. The inhibitor further includes two moieties of SEQ ID NO:1, two PEG₄ moieties, once cholesterol tag, and a pharmaceutically acceptable excipient. In some embodiments, each PEG₄ is flanked by a SEQ ID NO: 1 on one end and the cholesterol tag on the other end.

In certain aspects, a SARS-COV-2 (COVID-19) antiviral composition includes a SARS-COV-2 (COVID-19) lipid-peptide fusion inhibitor. The inhibitor further includes one moiety of SEQ ID NO:1, one PEG₄ moiety, once cholesterol tag, and a pharmaceutically acceptable excipient. In some embodiments, the PEG₄ is flanked by a SEQ ID NO: 1 on one end and the cholesterol tag on the other end.

In certain aspects, the invention provides a method of preventing COVID-19 that includes administering to a subject in need an antiviral pharmaceutical composition. The pharmaceutical composition includes a peptide where the C-terminal part of the peptide is “Gly-Ser-Gly-Ser-Cys,” and the N-terminal part of the peptide is selected from SEQ ID NO:1 and SEQ ID NO:2, or a peptide where the C-terminal part of the peptide is “Gly-Ser-Gly-Ser-Cys,” and the N-terminal part of the peptide has more than 80%, 85%, 90%, 95%, but less than 100% homology with a sequence selected from SEQ ID NO:1 and SEQ ID NO:2, a lipid tag, a spacer, and a pharmaceutically acceptable excipient.

In some embodiments, the lipid tag is Cholesterol, Tocopherol, or Palmitate.

In certain aspects, the invention provides a method of preventing COVID-19 that includes administering to a subject in need an antiviral pharmaceutical composition. The pharmaceutical composition includes a SARS-COV-2 (COVID-19) lipid-peptide fusion inhibitor, which further includes two moieties of SEQ ID NO:1, two PEG₄ moieties, one cholesterol tag, and a pharmaceutically acceptable excipient, wherein each PEG₄ is flanked by SEQ ID NO: 1 on one end and cholesterol on the other end.

In certain aspects, the invention provides a method of preventing COVID-19 that includes administering to a subject in need an antiviral pharmaceutical composition. The pharmaceutical composition includes a SARS-COV-2 (COVID-19) lipid-peptide fusion inhibitor, which further includes one moiety of SEQ ID NO: 1, one PEG₂₄ moiety, one cholesterol tag, and a pharmaceutically acceptable excipient, wherein the PEG₂₄ is flanked by SEQ ID NO: 1 on one end and cholesterol on the other end.

In some embodiments, the antiviral pharmaceutical composition is administered per airway or subcutaneously. In some embodiments, the antiviral pharmaceutical composition is administered intranasally. In some embodiments, the antiviral pharmaceutical composition is administered as nasal drops or a spray. In some embodiments, the antiviral pharmaceutical composition is administered as nasal powder.

In some embodiments, the antiviral pharmaceutical composition is administered to the subject at least two times. In some embodiments, at least one administration occurs before the subject is exposed to SARS-COV-2. In some embodiments, all administrations occur before the subject is exposed to SARS-COV-2. In some embodiments, the antiviral pharmaceutical composition is administered daily.

In some embodiments, the antiviral pharmaceutical composition is administered to the subject once. In some embodiments, the administration occurs before the subject is exposed to SARS-COV-2.

In some embodiments, the antiviral pharmaceutical composition is administered to the subject in need thereof with one or more additional antiviral substances. In some embodiments, at least one additional antiviral substance targets a different aspect of SARS-CoV-2 life cycle than SARS_{HRC} peptides.

In some embodiments, the peptide reaches biologically effective concentrations both in upper and lower respiratory tract of the subject. In some embodiments, the peptide reaches biologically effective concentrations in the lungs of the subject. In some embodiments, the peptide reaches biologically effective concentrations in the blood of the subject.

In some embodiments, the method prevents COVID-19 caused by SARS-COV-2 virions that comprise a Spike protein, wherein the sequence of the Spike protein differs from SEQ ID NO:3. In some embodiments, the SARS-COV-2 is selected from the group consisting of SARS-COV-2 S247R, SARS-COV-2 D614G, SARS-COV-2 S943P, and SARS-CoV-2 D839Y. In some other embodiments, the SARS-COV-2 is selected from the group consisting of SARS-COV-2 alpha, beta, gamma, delta, and lambda variants.

In certain aspects, the invention provides a method of reducing the risk of a SARS-COV-2 infecting a cell in a subject. The method includes administering an effective amount of a SARS-COV-2 (COVID-19) antiviral composition to inhibit SARS-COV-2 infection of a cell. The SARS-COV-2 (COVID-19) antiviral composition includes a SARS-CoV-2 (COVID-19) lipid-peptide fusion inhibitor comprising two moieties of SEQ ID NO:1, two PEG₄ moieties, one cholesterol tag, and a pharmaceutically acceptable excipient. Each PEG₄ can be flanked by a SEQ ID NO: 1 on one end and the cholesterol tag on the other end. Alternatively, The SARS-COV-2 (COVID-19) antiviral composition includes a SARS-COV-2 (COVID-19) lipid-peptide fusion inhibitor comprising one moiety of SEQ ID NO:1, one PEG₂₄ moiety, one cholesterol tag, and a pharmaceutically acceptable excipient. PEG₂₄ can be flanked by a SEQ ID NO: 1 on one end and the cholesterol on the other end.

In certain aspects, the invention provides a method of reducing the risk of COVID-19 in a subject. The method includes administering an effective amount of a SARS-CoV-2 (COVID-19) antiviral composition to inhibit SARS-COV-2 infection of a cell. The SARS-COV-2 (COVID-19) antiviral composition includes a SARS-COV-2 (COVID-19) lipid-peptide fusion inhibitor comprising two moieties of SEQ ID NO:1, two PEG₄ moieties, one cholesterol tag, and a pharmaceutically acceptable excipient. Each PEG₄ can be flanked by a SEQ ID NO: 1 on one end and the cholesterol tag on the other end. Alternatively, The SARS-COV-2 (COVID-19) antiviral composition includes a SARS-COV-2 (COVID-19) lipid-peptide fusion inhibitor comprising one moiety of SEQ ID NO: 1, one PEG₂₄ moiety, one cholesterol tag, and a pharmaceutically acceptable excipient. PEG₂₄ can be flanked by a SEQ ID NO: 1 on one end and the cholesterol on the other end.

In certain aspects, the invention provides a method of reducing the risk of death from COVID-19 in a subject. The method includes administering an effective amount of a SARS-COV-2 (COVID-19) antiviral composition to inhibit SARS-COV-2 infection of a cell. The SARS-COV-2 (COVID-19) antiviral composition includes a SARS-COV-2 (COVID-19) lipid-peptide fusion inhibitor comprising two moieties of SEQ ID NO:1, two PEG₄ moieties, one cholesterol tag, and a pharmaceutically acceptable excipient. Each PEG₄ can be flanked by a SEQ ID NO: 1 on one end and the cholesterol tag on the other end. Alternatively, The SARS-COV-2 (COVID-19) antiviral composition includes a SARS-COV-2 (COVID-19) lipid-peptide fusion inhibitor comprising one moiety of SEQ ID NO: 1, one PEG₂₄ moiety, one cholesterol tag, and a pharmaceutically acceptable excipient. PEG₂₄ can be flanked by a SEQ ID NO: 1 on one end and the cholesterol on the other end.

In some embodiments, the method prevents COVID-19 caused by SARS-COV-2 virions that comprise a Spike protein, wherein the sequence of the Spike protein differs from SEQ ID NO:3. In some embodiments, the SARS-COV-2 is selected from the group consisting of SARS-COV-2 S247R, SARS-COV-2 D614G, SARS-COV-2 S943P, and SARS-CoV-2 D839Y. In some other embodiments, the SARS-COV-2 is selected from the group consisting of SARS-COV-2 alpha, beta, gamma, delta, and lambda variants.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: General Concept

### Coronavirus Infection

Coronaviruses (CoVs) can cause life-threatening diseases. The latest disease was named coronavirus disease 2019 (abbreviated “COVID-19”) by the World Health Organization. COVID-19 is caused by the coronavirus strain SARS-COV-2. Like its predecessors SARS-COV-1 and middle eastern respiratory syndrome virus MERS-COV, SARS-COV-2 is a betacoronavirus. However, SARS-COV-2 and COVID-19 differ from the other CoVs (such as MERS) and their respective diseases in striking manners, as witnessed by the entire world in 2020.

Coronavirus Entry Pathway into Target Cells

Coronaviruses employ a type I fusion mechanism to gain access to the cytoplasm of host cells. Other pathogenic viruses that employ the type I fusion mechanism include HIV, paramyxoviruses and pneumoviruses. Merger of the viral envelope and host cell membrane is driven by profound structural rearrangements of trimeric viral fusion proteins; infection can be arrested by inhibiting the rearrangement process.

Infection by coronavirus requires membrane fusion between the viral envelope and the cell membrane. Depending on the cell type and the coronavirus strain, fusion can occur at either the cell surface membrane or in the endosomal membrane. The fusion process is mediated by the viral envelope glycoprotein (S), a ˜1200 residue, heavily glycosylated type-I integral membrane protein presented as a large homotrimer, each monomer having several domains (FIGS. 1, 2). A receptor binding domain (RBD)—distal to the viral membrane—is responsible for cell surface attachment. Membrane merger is mediated by a proximal cell fusion domain (FD). Concerted action by the RBD and FD is required for fusion. Upon viral attachment (and uptake in certain cases), host factors (receptors and proteases) trigger large scale conformational rearrangements in the FD, driven by formation of an energetically stable 6-helix bundle (6HB) that couples protein refolding directly to membrane fusion. The FD is thought to form a transient pre-hairpin intermediate composed of a highly conserved trimeric coiled-coil core that can be targeted by fusion inhibitory peptides (referred to as C-terminal heptad repeat, C-peptides, or HRC peptides).

Like the influenza HA, the S protein exists as a trimer on the virion surface and mediates attachment, receptor binding and membrane fusion. The betacoronaviruses S proteins' host cell receptors identified thus far include angiotensin-converting enzyme 2 (ACE2) for SARS-COV-1 and dipeptidyl peptidase-4 (DPP4) for MERS-COV. SARS-COV-2 was found to use the human angiotensin-converting enzyme 2 (hACE2) for entry (and most likely uses or can use other receptors as yet unknown). S undergoes cleavage by a host protease to generate S₁ and S₂. Priming with the receptor and cleavage are both necessary for membrane merger

### Pathways of Viral Entry and Strategies for Inhibition

The activation step that initiates a series of conformational changes in the fusion protein leading to membrane merger differs depending on the pathway that the virus uses to enter the cell. For many paramyxoviruses, upon receptor binding, the attachment glycoprotein activates the fusion protein to assume its fusion-ready conformation at the cell surface at neutral pH. We and others have shown that for these viruses (that fuse at the cell membrane), C-peptides derived from the HRC region of the fusion protein ectodomain inhibit viral entry with varying activity and that lipid conjugation markedly enhances their antiviral potency and simultaneously increases their in vivo half-life. By targeting lipid-conjugated fusion inhibitory peptides to the plasma membrane, and by engineering increased HRN-peptide binding affinity, we have increased antiviral potency by several logs. The lipid-conjugated inhibitory peptides on the cell surface directly target the membrane site of viral fusion. By adding poly-ethylene glycol (PEG) linkers (such as PEG₄) to the compounds between the lipid moiety and the peptide, we further increased the activity and potency of the conjugates. We demonstrated in vivo efficacy of lipid-conjugated fusion inhibitory peptides against lethal Nipah virus infection in golden hamsters and non-human primates, measles virus infection in mice and cotton rats, and human parainfluenza virus type 3 infection in cotton rats.

For viruses that do not fuse at the cell membrane the target for C-peptides is generally thought to be inaccessible. Example of these viruses are influenza and Ebola viruses. The fusion proteins of influenza (hemagglutinin protein; HA) and of Ebola (GP) are activated to fuse only after intracellular internalization. We showed that our lipid-conjugated peptides derived from influenza HA inhibit infection by influenza, suggesting that the lipid-conjugation-based strategy permits the use of fusion-inhibitory peptides for viruses that fuse in the cell interior. A second strategy that we adopted for influenza is the addition of HIV-TAT (a well known cell-penetrating peptide, CPP) to enhance inhibition of intracellular targets. With the combination of these two strategies, HA derived peptides are effective in vivo against human strains of influenza virus. A similar strategy also led to effective antiviral C-peptides for Ebola infection.

### Proof of Principle: Fusion Lipid-Peptides

A major challenge in developing C-peptide fusion inhibitors for coronavirus may be that coronavirus viral entry can follow several entry pathways (FIG. 2). Some coronavirus strains can fuse at the cell surface, however several others initially endocytose, and fusion is triggered in the endosome. In some cases, the same strain, depending on the S cleavage site and the target host cell protease, can enter via different pathways. The virus can fuse on the cell surface or inside the cells.

For this reason, design of entry inhibitors for coronavirus is a challenge. We explored whether adding cell penetrating peptides and lipid moieties that promote endosomal localization would increase the antiviral potency.

Earlier research on lipid-conjugated inhibitory peptides demonstrated that the lipid directs the peptide to cell membranes and increases antiviral efficacy. These conjugated peptides were shown, in published work, to inhibit both early and late entry strains of coronavirus (FIG. 3).

### Example 2: Design of the HRC-Derived SARS_{HRC} (Also Named SARS) and SARSMod Antiviral Peptides

### Sequence of the HRC Domain of the SARSCOV-2 S Protein

The SARS-COV-2 6HB assembly (FIG. 4) provides an excellent basis for design of inhibitors of SARS-COV-2 membrane fusion. The HRC domain features a central five-turn α-helix and extended regions flanking the helix on both sides. The native HRC domain corresponds to residues 1168-1203 of the SARS-COV-2 S protein.

Peptide SARS (FIG. 5) corresponds to the SARS-COV-2 HRC domain (identical to the SARS-COV-1 HRC domain); Xia et al. recently reported that peptide SARS (also named “D-1” or “peptide D-1”) is a modest inhibitor of SARS-COV-2 infection in a pseudovirus-based cellular assay (IC₅₀˜1 μM). Residues that form the central α-helix are indicated. Proposed SARSMod contains seven amino acid changes relative to SARS (shown as highlighted in FIG. 5) to improve solubility.

### Design of the SARS and SARSMod Lipid Fusion Peptides

Infection by SARS-COV-2 requires membrane fusion between the viral envelope and the host cell membrane, at either the cell surface or the endosomal membrane. The fusion process is mediated by the viral envelope spike glycoprotein, S. Upon viral attachment or uptake, host factors trigger large-scale conformational rearrangements in S, including a refolding step that leads directly to membrane fusion and viral entry. Peptides corresponding to the highly conserved heptad repeat (HR) domain at the C-terminus of the S protein (HRC peptides) may prevent this refolding and inhibit fusion, thereby preventing infection.

We recently described a monomeric SARS-COV-2 HRC-lipopeptide fusion inhibitor against SARS-COV-2 with in vitro and ex vivo efficacy superior to previously described HRC-derived fusion inhibitory peptides. We designed a number of constructs based on the SARS_{HRC} (also named SARS) and SARSMod peptide sequences. Basically, the SARS and SARSMod peptides were modified by attaching a glycine-serine 4-mer, GSGS, and a cysteine at their C-terminals. A PEG linker (PEG₄, PEG₂₄, or PEG₁₁) and a cholesterol tag were further added to the constructs. The HRC peptides form six-helix bundle (6HB)-like assemblies with the extended intermediate form of the S protein trimer, thereby disrupting the structural rearrangement of S that drives membrane fusion.

[SARS_{HRC}-PEG₄]-Chol (Also Named “SARS Monomer”):
SARS-GSGS-C-PEG₄-Chol

### [SARSMod-PEG₄]-Chol (Also Named “SARSMod Monomer”):

SARSMod-GSGS-C-PEG₄-Chol
   [SARS_{HRC}-PEG₄]₂-Chol (Also Named “SARS Dimer”):
[SARS-GSGS-C-PEG₄]₂-Chol
   [SARSMod-PEG₄]₂-Chol (Also Named “SARSMod Dimer”):
[SARSMod-GSGS-C-PEG₄]₂-Chol

We also designed additional constructs as variations from the constructs above. The design of the peptides is demonstrated in FIG. 6C. The identity of the conjugates was verified by MALDI-TOF MS, as shown in FIG. 7.
SARS_{HRC}-Chol: SARS-Chol (no linker)
[SARS_{HRC}]₂-PEG₁₁: dimer of SARS with no cholesterol, only PEG₁₁
SARS_{HRC}-PEG₂₄-chol: monomer of SARS with PEG₂₄ and cholesterol

We have previously demonstrated that lipid conjugation of HRC-derived inhibitory peptides markedly increases antiviral potency and in vivo half-life, and successfully used this strategy to create entry inhibitors for prophylaxis and/or treatment of human parainfluenza virus type 3, measles virus, influenza virus, and Nipah virus infection. Both dimerization and peptide integration into cell membranes proved key to ensure respiratory tract protection and prevent systemic lipopeptide dissemination. The lipid-conjugated peptides administered intranasally to animals reached high, and biologically effective (in vivo) concentrations both in the upper and lower respiratory tract, and the specific nature of the lipid can be designed to modulate the extent of transit from the lung to the systemic circulation and organs. Lipid conjugation also enabled activity against viruses that do not fuse until they have been taken up via endocytosis. Here, we show that a SARS-CoV-2 S-specific lipopeptide is a potent inhibitor of fusion, prevents viral entry, and, when administered intranasally, completely prevents direct-contact transmission of SARS-COV-2 in ferrets. We propose this compound as a candidate antiviral, for pre-exposure or early post-exposure prophylaxis for SARS-COV-2 transmission in humans.

### Example 3: In Vitro Potency of the SARS and SARSMod Derived Lipid-Peptide Fusions

To improve the antiviral potency of the previously assessed SARS-COV-2 HRC-lipopeptide fusion inhibitor, we compared monomeric and dimeric derivatives of the SARS-CoV-2 S-derived HRC-peptide (FIG. 8). Initial functional evaluation of the SARS-COV-2 HRC lipopeptides was conducted with a cell-cell fusion assay based on alpha complementation of β-galactosidase (β-gal) that we adapted for assessment of SARS-COV-2 S-mediated fusion.

FIG. 8A shows the antiviral potency of four monomeric and two dimeric SARS-CoV-2 S-derived 36-amino acid (FIGS. 5 and 6) HRC-peptides, without (SARS_{HRC} and [SARS_{HRC}]₂-PEG₁₁) or with appended cholesterol, in quantitative cell-cell fusion assays. The percentage inhibition corresponds to the extent of luminescence signal suppression observed in the absence of any inhibitor (i.e., 0% inhibition corresponds to maximum luminescence signal). Dimerization increased the peptide potency for both non-lipidated peptides and their lipidated counterparts (FIG. 8A). A dimeric cholesterol-conjugated lipopeptide based on the HPIV3 F protein HRC domain, used as a negative control, did not inhibit fusion at any concentration tested (black line in FIG. 8A, see FIG. 11b-c for additional negative controls). Among the monomeric lipopeptides, the peptide bearing PEG₂₄ was most potent. The dimeric cholesterol-conjugated peptide ([SARS_{HRC}-PEG₄]₂-chol; red line in FIG. 8A) is the most potent lipopeptide against SARS-COV-2 among the tested panel.

Despite the overall stability of the SARS-COV-2 genome, variants with mutations in S have spread globally. These mutations in S altered infectivity of cells (e.g., D614G) or were located in the putative target domain of the HRC peptide (e.g., S943P). To determine the potency of the [SARS_{HRC}-PEG₄]₂-chol peptide for a range of variant SARS-COV-2 viruses, we examined fusion inhibition mediated by each of these emerging S protein mutants. In addition, to assess the potential for broad-spectrum activity, we assessed potency against the S of SARS-COV and MERS-COV (using dipeptidyl peptidase 4 (DPP4) receptor-bearing cells as the target for the latter). The dimeric cholesterol-conjugated peptide ([SARS_{HRC}-PEG₄]₂-chol) also robustly inhibited fusion mediated by the S proteins of several emerging SARS-COV-2 variants (including D614G) and the S protein of SARS-COV and MERS-COV (FIG. 8B). Lastly, as long as the coronavirus disease 2019 (COVID-19) pandemic is ongoing, the genome of the causative agent, SARS-COV-2, is continuously evolving. Many changes are transient or of no epidemiological or clinical impact, but multiple variants emerged that are classified as variant of interest (VOI) or variants of concern (VOC). Here, to specifically address two VOC, the British variant or the alpha variant (B.1.1.7; UK) and the South African variant or the beta variant (B.1.351; SA), (https://www.cdc.gov/coronavirus/2019-ncov/more/science-and-research/scientific-brief-emerging-variants.html, Muik 2021, and Wu 2021. For nomenclature please see https://www.who.int/en/activities/tracking-SARS-COV-2-variants/) the experiment was repeated with the variants alpha (B.1.1.7) and beta (B.1.351) (FIG. 8C). We concluded that the [SARS_{HRC}-PEG₄]₂-chol peptide robustly inhibited fusion mediated by the S proteins of these two variants as well.

HIV-TAT is a well-known cell-penetrating peptide (CPP) to enhance inhibition of intracellular targets. We have previously shown that the addition of cell penetrating peptide sequence can increase the antiviral activity for both peptides targeting Ebola and Influenza viruses. For influenza, only the TAT conjugated peptides were shown to be effective in vivo. Surprisingly, addition of cell penetrating peptide sequence does not increase the antiviral activity of [SARS_{HRC}-PEG₄]₂-chol, as illustrated in FIG. 9. As shown in FIG. 9A, the TAT addition decreases efficacy in VeroE6, which is unexpected finding since in viruses that fuse inside the endosome, one expects TAT to enhance—like for Ebola. In these cells without TMPRSS2 endosome pathway fusion was expected and therefore it is unexpected that TAT makes efficacy worse. As shown in FIG. 9B, the TAT also decreases efficacy in VeroE6-TMPRSS2 cells, although we did not expect it to enhance here since in the presence of TMPRSS2 this virus should fuse at the cell surface. This surprising result further emphasizes the differences between the different viruses and how they react to peptide inhibitors.

Proposed anchoring of the dimeric lipopeptide in the host cell membrane and interactions with the viral S protein are shown in FIG. 10A-B. Our SARS-COV-2 S-derived HRC-peptides demonstrated surprising and unexpectedly high potency in vitro. Collectively, these data suggest that particularly the [SARS_{HRC}-PEG₄]₂-chol lipopeptide is equipped to combat an evolving pandemic. The design and specificity of SARS-COV-2 inhibition by this peptide is illustrated in further details in FIG. 11.

### Example 4: Biodistribution, Cellular Cytotoxicity, and Virus Entry Blocking of the SARS and SARSMod Derived Lipid-Peptide Fusions

For other enveloped respiratory viruses, we previously showed that both ex vivo and in vivo dimeric lipopeptides administered intranasally displayed different retentions in the respiratory tract dependent on the attached moiety of chol versus toc (Figueira, T. N. et al. J Virol 91 (2017)). Here, we compared local and systemic biodistribution of our most potent monomeric and dimeric lipopeptides (SARS_{HRC}-PEG₂₄-chol and [SARS_{HRC}-PEG₄]₂-chol) at 1, 8, and 24 hours after intranasal inoculation or subcutaneous injection in humanized K18 hACE2 mice (FIGS. 12-14). The two lipopeptides reached a similar lung concentration at 1 hour after intranasal administration (˜1-2 μM). At 8 and 24 hours, the dimeric [SARS_{HRC}-PEG₄]₂-chol lipopeptide remained at high levels in the lung with minimal entry into the blood, but the monomeric peptide entered the circulation and the lung concentration decreased (FIG. 12). These high levels in the lung are surprising and unexpected; they are effective in animals and expected to be clinically effective. The dimeric [SARS_{HRC}-PEG₄]₂-chol lipopeptide was distributed throughout the lung after intranasal administration (FIG. 13). FIG. 14 further depicts the distribution of both lipopeptides in different tissues and at a longer time point (48 hours).

An ex vivo toxicity (MTT) assay in primary HAE cells was conducted for the fusions. The assay showed minimal toxicity even after 6 days at the highest concentrations tested (<20% at 100 μM), and no toxicity at its IC₉₀ entry inhibitory concentrations (˜35 nM) (FIG. 15). The longer respiratory tract persistence of [SARS_{HRC}-PEG₄]₂-chol, in concert with its in vitro efficacy, led us to advance this dimeric lipopeptide for further assessments.

Next, the lead peptide, [SARS_{HRC}-PEG₄]₂-chol, was assessed for its ability to block entry of live SARS-COV-2 in VeroE6 cells or VeroE6 cells overexpressing the protease TMPRSS2, one of the host factors thought to facilitate viral entry at the cell membrane. Whereas viral fusion in VeroE6 cells predominantly occurs after endocytosis, the virus enters TMPRSS2-overexpressing cells by fusion at the cell surface, reflecting the entry route in airway cells. This difference is highlighted by chloroquine's effectiveness against SARS-CoV-2 infection in Vero cells but failure in TMPRSS2-expressing Vero cells and human lung. The [SARS_{HRC}-PEG₄]₂-chol peptide dissolved in an aqueous buffer containing 2% dimethylsulfoxide (DMSO) inhibited virus entry after 8 hours with an IC₅₀˜300 nM in VeroE6 and ˜5 nM in VeroE6-TMPRSS2 cells (FIG. 16A). To strengthen translational potential towards human use, the lipopeptide was reformulated in sucrose instead of DMSO, resulting in equivalent potency (FIG. 16B). A control dimeric fusion-inhibitory lipopeptide directed against HPIV3 blocked infection by HPIV3 (green line), but did not inhibit SARS-CoV-2 infection. The in vitro efficacy data are summarized in FIG. 16C.

### Example 5: Potency of Fusion Inhibitory Lipopeptides Against SARS-COV-2 Variants of Concern in Comparison to Monoclonal Antibodies and Post-Vaccination Sera

Here, we determined and characterize the potency of fusion inhibitory lipopeptides against two VOC, the alpha (B.1.1.7) variant and the South African or beta (B.1.35) variant. For both VOC, immune escape from monoclonal antibodies has been described and convalescent sera are reported as less potent against B.1.351 in neutralization assays.

We have previously described that wt SARS-COV-2 infection can be efficiently inhibited by fusion inhibitory lipopeptides in vitro and in vivo. Here, we evaluated the efficacy of two lipopeptides ([SARS_{HRC}-PEG₄]₂-chol and SARS_{HRC}-PEG₂₄-chol) against wild type, the alpha variant (B.1.1.7) and the beta variant (B.1.351) SARS-COV-2 in an infectious virus entry assay. We directly compared the lipopeptides to a set of eleven previously described monoclonal antibodies (mAb) and eight post-vaccination sera (BNT162b2, two shots).

We performed a previously established infectious virus entry assay. Briefly, FIP, mAb and sera were serially diluted (10-fold, 5-fold or 2-fold, respectively) and incubated with a fixed amount of virus particles for one hour at 37 C. Virus-inhibitor mix was subsequently added to VeroE6 cells overexpressing TMPRSS2 (VeroE6-TMPRSS2) or Calu3 cells and incubated for eight hours at 37 C. Cells were washed, fixed and stained with a primary mouse-anti-SARS-COV nucleocapsid (Biorad) and a secondary goat-anti-mouse IgG/FITC antibody (Invitrogen). Fluorescent spots were recorded, counted and inhibition was calculated in percentage of infection control. We calculated IC₅₀ values using a four-parameter dose response model and defined potencies within each class based on log-transformed IC₅₀ values (shown in FIGS. 17 and 18, both depicting the same results in different format).

We detected comparable potency of both lipopeptides against wt, the alpha variant (B.1.1.7) and the beta variant (B.1.351), independent of the cell line used. On VeroE6-TMPRSS2 cells 2 out of 11 mAb inhibited viral entry for all three viruses efficiently (2-15, 1-16); 4 out of 11 mAb did not (or only at high concentrations) inhibit viral entry; 2 out of 11 mAb efficiently inhibited wt SARS-COV-2 entry but not the alpha variant (B.1.1.7) or the beta variant (B.1.351) entry (1-21, 1-22); 2 out of 11 mAb blocked wt and the alpha variant (B.1.1.7) entry at comparable levels but did not block the beta variant (B.1.351) entry (1-18, 217); and 1 out of 11 mAb had slightly increased efficacy to the alpha variant (B.1.1.7) (2-02). Although IC₅₀ values were in general lower on Calu3 cells, we observed a similar trend.

Polyclonal post-vaccination sera showed a broad spectrum of reactivity against all variants. However, compared to wt SARS-COV-2, we measured overall lower titers to the beta variant (B.1.351) and comparable or higher titers to the alpha variant (B.1.1.7). FIG. 19 shows entry inhibition plots for lipopeptides (A), mAb (B-D) and postvaccination sera (E-G) on VeroE6-TMPRSS cells. mAb were categorized based on activity for different variants (B: comparable activity to all three viruses, C: activity against wt SARS-COV-2, D: no activity). FIG. 20 further demonstrates Fusion inhibitory activity of [SARS_{HRC}-PEG₄]₂-chol peptide against different SARS-COV-2 S variants. These include the alpha, beta, gamma, delta, and lambda variants.

In conclusion, we show equal efficacy of the evaluated SARS-COV-2 specific lipopeptides against wt SARS-COV-2 and the VOC the alpha variant (B.1.1.7) and the beta variant (B.1.351). Furthermore, we confirmed immune escape from multiple well-characterized mAb by at least one of the tested VOC, as well as lower serum titers against the beta variant (B.1.351).

### Example 6: Experiment Design and Methodology of In Vivo Potency Test of [SARS-PEG₄]₂-chol

Ferrets are an ideal model for assessing respiratory virus transmission, either by direct contact or by aerosol transmission. Mustelids are highly susceptible to infection with SARS-COV-2, as also illustrated by frequent COVID-19 outbreaks at mink farms. Direct contact transmission of SARS-COV in ferrets was demonstrated in 2003, and both direct contact and airborne transmission have been shown in ferrets for SARS-COV-22. Direct contact transmission in the ferret model is highly reproducible (100% transmission from donor to acceptor animals), but ferrets display limited clinical signs. After infection via direct inoculation or transmission, SARS-COV-2 can readily be detected in and isolated from the throat and nose, and viral replication leads to seroconversion.

To assess the efficacy of [SARS_{HRC}-PEG₄]₂-chol in preventing SARS-COV-2 transmission, naive ferrets were dosed prophylactically with the lipopeptide before being co-housed with SARS-COV-2 infected ferrets. In this setup, transmission via multiple routes can theoretically occur (aerosol, orofecal, and scratching or biting), and ferrets are continuously exposed to infectious virus during the period of co-housing, providing a stringent test for antiviral efficacy. The study design is shown in FIG. 21A. Three donor ferrets (grey in diagram) were inoculated intranasally with 5×105 TCID50 SARS-COV-2 on day 0. Twelve recipient ferrets housed separately were treated by nose drops with a mock preparation (red) or [SARS_{HRC}-PEG₄]₂-chol peptide (green) on 1- and 2-days post-inoculation (DPI) of the donor animals. The [SARS_{HRC}-PEG₄]₂-chol peptides for intranasal administration were dissolved to a concentration of 6 mg/mL in an aqueous buffer containing 2% DMSO, administering a final dose of 2.7 mg/kg to ferrets (450 uL, equally divided over both nostrils). Peptide stocks and working dilutions had similar IC50's, confirming that peptide-treated ferrets were dosed daily with comparable amounts (FIG. 12A-B). Six hours after the second treatment on 2 DPI, one infected donor ferret (highly positive for SARS-COV-2 by RT-qPCR) was co-housed with four naive recipient ferrets (two mock-treated, two peptide-treated). After a 24-hour transmission period in three separate, negatively pressurized HEPA-filtered ABSL3-isolator cages, co-housing was stopped and donor, mock-treated and peptide-treated ferrets were housed as separate groups. Additional [SARS_{HRC}-PEG₄]₂-chol peptide treatments were given to recipient animals on 3 and 4 DPI.

The viral loads (detection of viral genomes via RT-qPCR) for directly inoculated donor animals (grey), mock-treated recipient animals (red) and lipopeptide-treated recipient animals (green) are shown in FIG. 21B-C. All directly inoculated donor ferrets were productively infected, as shown by SARS-COV-2 genome detection in throat and nose swabs, and efficiently and reproducibly transmitted the virus to all mock-treated acceptor ferrets (FIG. 21B-C, red curves). Productive SARS-COV-2 infection was not detected in the throat or nose of any of the peptide-treated recipient animals (FIG. 21B-C, green curves). A slight rise in viral loads in samples collected at 3 DPI was detected (at the end of the co-housing), confirming that peptide-treated animals were exposed to SARS-COV-2. In FIG. 21D 16d the area under the curve (AUC) shows the striking difference between the mock treated and the peptide treated animals. No infectious virus was isolated from lipopeptide-treated ferrets, while infectious virus was detected in all mock-treated ferrets (FIG. 21E). Virus isolation data correlated with genome detection (FIG. 21F).

Seroconversion occurred in donor ferrets and 6/6 mock-treated animals by 21 DPI, but in none of the peptide-treated recipient animals, as shown by S- and N-specific IgG enzyme-linked immunosorbent assay (ELISA) and virus neutralization (FIG. 21G-I). Successful challenge infection confirmed that in-host virus replication had been completely blocked by the [SARS_{HRC}-PEG₄]₂-chol treatment (FIG. 21J and FIG. 23) and that none of the peptide-animals were protected, whereas the mock-treated animals (which had seroconverted) were all protected. Collectively, these data show that, surprisingly and unexpectedly, intranasal prophylactic administration of the [SARS_{HRC}-PEG₄]₂-chol peptide had protected 6/6 ferrets from transmission and productive infection.

### Example 7: Single Administration of the Dimeric Lipopeptide

In light of the persistence of the dimeric lipopeptide in the murine lung (FIG. 12 and FIG. 13), we assessed the potential for a single administration of sucrose-formulated lipopeptide in a ferret transmission experiment two hours before co-housing to prevent or delay infection. In this experiment, we used a dimeric HPIV3-specific lipopeptide as mock control (FIG. 24). Although sucrose formulation had resulted in promising results in vitro at small scale (FIG. 16B), formulation at larger scale resulted in incomplete dissolution. As a consequence, the sucrose-formulated [SARS_{HRC}-PEG₄]₂-chol lipopeptide was administered at a substantially lower concentration than in the experiment with the DMSO-formulated lipopeptide (FIG. 22C-D). Nevertheless, surprisingly and unexpectedly, the SARS-COV-2 lipopeptide provided a significant level of protection as compared to the HPIV3 control group, and four out of six SARS-COV-2 lipopeptide-treated animals were protected against infection. This experiment suggests that single-administration pre-exposure prophylaxis is promising, while the optimal formulation and dosing regimen is an area of ongoing experimentation.

The intranasal [SARS_{HRC}-PEG₄]₂-chol peptide presented in this study is the first successful prophylaxis that prevents SARS-COV-2 transmission in a relevant animal model, providing complete protection during a 24-hour period of intense direct contact. Parallel approaches to prevent transmission that target the interaction between S and ACE2 have shown promise in vitro (e.g., the “miniprotein” approach). The lipopeptide described here acts on the S2 domain after shedding of S1 (FIGS. 10A-B), and is complementary to strategies that target S1's functions or maintain S in its pre-fusion conformation, e.g. synthetic nanobodies. Fusion-inhibitory lipopeptides could be used for pre- and post-exposure prophylaxis in combination with these strategies, and in conjunction with treatments (e.g., ribonucleoside analogs) that reduce replication in a treated infected individual. A combination of drugs that target different aspects of the viral life cycle is likely ideal for this rapidly-evolving virus. The [SARS_{HRC}-PEG₄]₂-chol peptide has a long shelf life, does not require refrigeration and can easily be administered, making it particularly suited to treating hard-to-reach populations. This is key in the context of COVID-19, which has reached every community with the burden falling disproportionately on low-income and otherwise marginalized communities. This HRC lipopeptide fusion inhibitor is feasible for advancement to human use and should readily translate into a safe and effective nasal spray or inhalation administered fusion inhibitor for SARS-COV-2 prophylaxis, supporting containment of the ongoing COVID-19 pandemic.

### Example 8: Materials and Methods

Ethics statement. Influenza virus, SARS-COV-2 and Aleutian Disease Virus seronegative female (weighing 900-1200 g) and male (weighing 1000-1500 g) ferrets (Mustela putorius furo) were obtained from a commercial breeder (Triple F Farms, PA, USA). Animals were housed and experiments were performed in compliance with the Dutch legislation for the protection of animals used for scientific purposes (2014, implementing EU Directive 2010/63). Research was conducted under a project license from the Dutch competent authority (license number AVD1010020174312) and the study protocol was approved by the institutional Animal Welfare Body (Erasmus MC permit number 17-4312-07, -08 and -09). Animal welfare was monitored on a daily basis. K18-hACE2 mice [B6. Cg-Tg(K18-hACE2)2Prlmn/J] (4-6 weeks old) were purchased from the Jackson Laboratory and bred in house (at CUIMC, NY, USA). All mouse experiments were conducted in accordance with protocols approved by the Columbia University Institutional Animal Care and Use Committee (AC-AABG9559). The Institute of Comparative Medicine (ICM) at Columbia University is fully accredited by the Association for the Assessment and Accreditation of Laboratory Animal Care, International (AAALAC) and complies with the regulations under the Animal Welfare Act (AWA), the Health Research Extension Act of 1985, and the National Research Council (NRC).

SARS-COV-2 S protein mediated fusion modeling. Molecular Maya (https://clarafi.com/tools/mmaya/) was used to model and simulate the inhibitory lipopeptide, the full-length SARS-COV 2 Spike (S) pre-fusion, pre-hairpin and post-fusion structures, using a combination of molecular mechanics force fields. The pegylated cholesterol, inhibitory peptides, and S protein respectively were parametrized using the MMFF94 (1), CHARMM C36 (2), and Martini (3) force fields. Simulations were run using Autodesk Maya's nucleus solver and additional restraints native to the nucleus solver (nConstraints) were used to stabilize the molecules during interactive steering.

To model the initial full-length pre-fusion S protein, we used the SARS-COV 2 S protein sequence from UniProt entry PODTC2, PDB 6XR8 (4) from the wild-type SARS-CoV 2 S protein and 2FXP (5) from the SARS-COV S protein. Remaining structural gaps were modeled using Molecular Maya's Modeling kit.

To model the S pre-hairpin intermediate, we aligned the post-fusion structure from PDB 6XRA (+) to our full-length pre-fusion model, considering only the central helix (CH) region in the alignment (residues 968-1035). Simulations were then run to progressively steer the HRN region (residues 910-985) towards the aligned post-fusion structure, leading to the extension of the CH coiled coil by HRN. The remaining regions of the model were restrained with elastic networks or position restraints to preserve local secondary structure. The fusion peptide region was then released from the elastic network and steered towards the host-cell membrane, resulting in the pre-hairpin model.

The post-fusion model was obtained by progressively steering the HRC region from the pre-hairpin model towards the post-fusion structure to obtain the HRC-HRN 6-helix bundle. During the transition, the position restraints on the C-terminal transmembrane domains of the S were translated to allow HRC reaching the steering targets, and the entire post-fusion structure target was reoriented to avoid overlaps with the viral membrane proxy.

Finally, to model the inhibitory lipopeptide's interaction with the S protein, its amino-acids were steered towards the matching HRC residues in the target post-fusion structure, while the cholesterol moiety was positioned on the host-cell membrane plane. The S's pre-hairpin to post-fusion transition was interrupted before sterical clashes between HRC and the inhibitory peptide were detected.

Lipopeptide synthesis. The peptide (SARS_{HRC}) corresponding to residues 1168-1203 of SARS-COV-2 S with a C-terminal-GSGSGC spacer sequence was prepared by solid phase peptide synthesis (SPPS). The SARS_{HRC} peptide was acetylated at the N-terminus and amidated at the C-terminus. The crude peptide was purified by reverse-phase high-performance liquid chromatography (HPLC) and characterized by matrix-assisted laser desorption ionization time of flight mass spectrometry (MALDI-TOF MS). SARS_{HRC}-chol,SARS_{HRC}-PEG₄-chol, SARS_{HRC}-PEG₂₄-chol, [SARS_{HRC}]₂-PEG₁₁, and [SARS_{HRC}-PEG₄]₂-chol were synthesized via chemoselective Thiol-Michael addition reactions between the terminal thiol group on the peptide cysteine residue and either bromoacetyl chol (with and without the indicated PEG linkers), maleimide functional PEG linkers or PEG-cholesterol linkers as previously described (6). Purification by HPLC and lyophilization yielded the peptide-lipid conjugates as white powders. The identity of the conjugates was verified by MALDI-TOF MS (FIG. 7).

Dissolving lipopeptides for use in experiments. [SARS_{HRC}-PEG₄]₂-chol was supplied as a white powder in aliquots of 10 mg. For in vivo experiments in ferrets, 10 mg of [SARS_{HRC}-PEG₄]₂-chol was dissolved in 33.3 μl DMSO, which was subsequently added to 1632.7 μl de-ionized H2O. This yielded a final aqueous solution of lipopeptide dissolved at a concentration of 6 mg/mL containing 2% DMSO. To obtain peptide dissolved in aqueous solution without DMSO, 100 mg/ml of [SARS_{HRC}-PEG₄]₂-chol or [HPIV3_{HRC}-PEG₄]₂-chol in DMSO (10 mg of peptide in 100 μl of DMSO) and 1 mg/ml of sucrose in sterile water were prepared. 10 μl of the peptide solution (1 mg) was added to 100 μl of sucrose (0.1 mg). Lyophilization of the peptide solution (DMSO+sucrose) was performed over-night and dry powder was resuspended in 50 μl of DI water to a final concentration of 20 mg/ml in water without any DMSO.

Plasmids. The cDNAs coding for hACE2 fused to the fluorescent protein Venus, dipeptidyl peptidase 4 (DPP4) fused to the fluorescent protein Venus, SARS-COV-2 S and the indicated S variants, SARS-COV S, and MERS-S(codon optimized for mammalian expression) were cloned in a modified version of the pCAGGS (with puromycin resistance for selection).

Virus. SARS-COV-2 (isolate BetaCoV/Munich/BavPat1/2020; kindly provided by Prof. Dr. C. Drosten) was propagated to passage 3 on VeroE6 cells in OptiMEM I (1×)+GlutaMAX (Gibco), supplemented with penicillin (10,000 IU/mL, Lonza) and streptomycin (10,000 IU/mL, Lonza) at 37° C. VeroE6 cells were inoculated at a multiplicity of infection (MOI) of 0.01. Supernatant fluid was harvested 72 hours post inoculation (HPI), cleared by centrifugation and stored at −80° C. All live virus work was performed in a Class II Biosafety Cabinet under BSL-3 conditions at Erasmus MC. HPIV3-GFP was commercially obtained from Viratree, propagated to passage 3 on Vero cells in DMEM supplemented with 10% foetal bovine serum (FBS), penicillin (10,000 IU/mL, Lonza) and streptomycin (10,000 IU/mL, Lonza) at 37° C.

Cells. Human embryonic kidney (HEK) 293T and Vero (African green monkey kidney) cells were grown in Dulbecco's modified Eagle's medium (DMEM; Invitrogen; Thermo Fisher Scientific) supplemented with 10% FBS and antibiotics in 5% CO₂ at 37ºC. VeroE6 (ATCC CRL-1586) and VeroE6-TMPRSS2 cells were grown in DMEM (Gibco) with 10% FBS, 2 mM L-glutamine (Gibco), 10 mM Hepes (Lonza), 1.5 mg/ml sodium bicarbonate (NaHCO₃, Lonza), penicillin (10,000 IU/mL) and streptomycin (10,000 IU/mL) (7).

β-Gal complementation-based fusion assay. We previously adapted a fusion assay based on alpha complementation of β-galactosidase (β-Gal) (8). In this assay, hACE2 receptor-bearing cells (or dipeptidyl peptidase 4 (DPP4) receptor-bearing cells for MERS-CoV-2 experiments) expressing the omega peptide of β-Gal are mixed with cells co-expressing SARS-COV or SARS-COV-2 glycoprotein S and the alpha peptide of β-Gal, and cell fusion leads to alpha-omega complementation. Fusion is stopped by lysing the cells and, after addition of the substrate (® The Tropix Galacto-Star™ chemiluminescent reporter assay system, Applied Biosystem), luminescence is quantified on a Tecan M1000PRO microplate reader.

HAE cultures & toxicity assay. The EpiAirway AIR-100 system (MatTek Corporation) consists of normal human-derived tracheal/bronchial epithelial cells that have been cultured to form a pseudostratified, highly differentiated mucociliary epithelium closely resembling that of epithelial tissue in vivo (9). HAE cultures were incubated at 37° C. in the presence or absence of 1, 10, or 100 μM concentrations of the different peptides, which were added to the feeding medium every 2 days for 7 days. Cell viability was determined on day 7. Cycloheximide (CHE, a protein synthesis inhibitor in eukaryotes) was used as positive control for toxicity. Cell viability was determined after 24 h using the Vybrant MTT Cell proliferation Assay Kit according to the manufacturer's guidelines. The absorbance was read at 540 nm using Tecan M1000PRO microplate reader.

Antibodies against S-derived HRC peptides (HRC_{SARS}). Polyclonal antibodies against linear epitopes of HRC_{SARS} were generated (Genscript) in rabbits and validated in our western blot and ELISA assays. Genscript provided a full report confirming epitope recognition by the antibodies in an ELISA. The purified sera were aliquoted and lyophilized (10-20 mg in sealed bottles). Several aliquots of the purified sera were conjugated to biotin. Lyophilized aliquots were kept at −80° ° C. Once an aliquot was re-suspended, multiple liquid aliquots (50-100 μl) were made and re-frozen)(−80° ° C.

Mouse biodistribution experiments. Intranasal inoculation (FIG. 12) mice (n=3 or 4) under anaesthesia with a mixture of ketamine/xylazine (100 mg/kg and 10 mg/kg, respectively) were subjected to intranasal installation of lipopeptides (5 μg/g) dissolved in 40 μl of water and 2% DMSO (20 μl each nostril) by a dropper. Subcutaneous injection (FIG. 12a, b): mice under anaesthesia with a mixture of ketamine/xylazine (100 mg/kg and 10 mg/kg, respectively) were injected with lipopeptides (5 μg/g) dissolved in 100 μl of water and 2% DMSO in the subcutaneous tissue between the scapulae. In both experiments, mice were uniquely identified using ear tags. After recovery from anesthesia, mice were returned to the animal house and humanely euthanized at 3 time points (1,8, and 24 h post dosing) by cervical dislocation under isofluorane anesthesia before harvesting the tissues for analysis (lungs and blood). Lungs were weighed, mixed in PBS (1:1, w/vol), and homogenized using a BeadBug™ microtube homogenizer. Samples were subsequently treated with acetonitrile/1% TFA (1:4, vol/vol) overnight on a rotor at 4° C. and centrifuged for 10 min at 8000 rpm. Lipopeptides could not be detected in the lungs after subcutaneous administration (FIG. 12a, b). After intranasal administration, [SARS_{HRC}-PEG₄]₂-chol showed superior retention in the respiratory tract (FIG. 12c, d).

ELISA for semi-quantitative peptide assessment. 96 well plates Maxisorp (Nunc) were coated overnight with purified rabbit anti-HRC_{SARS} antibodies in carbonate/bicarbonate buffer (pH=7.4, 20 μg/ml). Plates were washed twice in 1×PBS and blocked in 3% BSA/1×PBS for 30 min. The blocking buffer was replaced by 2 dilutions of each sample in 3% BSA/1×PBS in duplicate and incubated for 1.5 h at room temperature (RT). Wells were washed 3× in 1×PBS, and developed with purified rabbit anti-HRC_{SARS} antibodies conjugated to biotin for 1 h at RT. Wells were washed 3 times in 1×PBS, developed using streptavidin conjugated to peroxidase in 3% BSA/1×PBS for 30′ at RT followed by 5 washes and incubation with Ultra TMB substrate solution (Sigma-Aldrich), and stopped with sulfuric acid (12%). Absorbance was read at 450 nm.

Immunohistochemical detection. Lung sections were de-paraffinized and blocked with 10% donkey serum in PBS for 1 h at RT. Rabbit anti-HRC_{SARS} antibody was added and incubated for 12 h at 4° C. Sections were stained with donkey anti-rabbit secondary antibody (Invitrogen, #A31572) for 1 hr at RT. Sections were treated with DAPI, mounted in Vectashield Mounting Medium (Vector Laboratories, Inc., Burlingame, CA), covered, and imaged with DMi8 (Leica Microsystems, Buffalo Grove, IL).

In vitro potency of fusion inhibitory lipopeptides. Potency of [SARS_{HRC}-PEG₄]₂-chol and [HPIV3_{HRC}-PEG₄]₂-chol was determined in an in vitro infectious virus fusion assay. Original stocks and working dilutions for animal experiments were tested in triplicate in VeroE6 and VeroE6-TMPRSS2 cells at concentrations of 0.06 nM to 5 μM (5-fold dilution series). Peptides were pre-incubated with the cells for 1 h at 37° C. After pre-incubation, SARS-COV-2 (600 TCID₅₀/well) was added. After 8 h at 37° C., cells were washed and fixed with 4% PFA for 20 min at RT. Plates were submerged in 70% ethanol and stained in a BSL-2 laboratory. In short, cells were washed with PBS and blocked with 10% normal goat serum (NGS) for 30 min at RT. Primary mouse-anti-SARS-COV nucleocapsid antibody (Biorad) was incubated for 1 h at RT in 10% NGS. After washing, secondary goat-anti-mouse IgG/FITC antibody (Invitrogen) was incubated for 45 min at RT in 10% NGS. Fluorescent spots were visualized with an Amersham Typhoon Biomolecular Imager (GE Healthcare) and counted with ImageQuant TL 7.0 software (GE Healthcare). The activity of [HPIV3_{HRC}-PEG₄]₂-chol against HPIV3 was determined using a similar assay. Original stocks were tested in triplicate in Vero cells at the same concentrations. After pre-incubation, rHPIV3-GFP (300 TCID₅₀/well) was added. Cells were washed and fixed with 2% PFA after 37 h and fluorescent spots were visualized and counted.

Ferret transmission experiment. For the experiment with DMSO-formulated lipopeptide: Three donor ferrets were inoculated intranasally with 5×10⁵ TCID₅₀/ml of SARS-COV-2 in 450 μl (225 μl instilled dropwise in each nostril) and were housed together in a negatively pressurized HEPA-filtered ABSL-3 isolator. This was considered the start of the experiment (0 days post inoculation, DPI). At the same time, twelve direct contact ferrets were divided over three other isolators. Ferrets were either mock-treated (vehicle, 2% DMSO in DI water) or treated with [SARS_{HRC}-PEG₄]₂-chol on 1-4 DPI. The peptide was inoculated intranasally in 450 μl (225 μl instilled dropwise in each nostril), HRC dimer-chol treated ferrets received a peptide dose of ˜2.7 mg/kg. Leftover batches were stored at −80° C. for later use in in vitro potency assays (FIG. 22A,B). At 2 DPI, six hours after the second treatment, one donor ferret was placed in the same isolators as two mock-treated and two peptide-treated ferrets, in three separate isolators. Each isolator now contained five ferrets, the donor ferret, the mock-treated recipient ferrets and the [SARS_{HRC}-PEG₄]₂-chol-treated recipient ferrets. At 3 DPI, 18 hours after onset of co-housing, the animals received a third mock or peptide treatment. Six hours later, i.e. 24 hours after the start of the co-housing, the donor animals were moved back to their original isolator and the mock-treated and peptide-treated ferrets were housed in two groups of six animals in clean isolators (FIG. 21A). Throat and nose swabs were collected from the animals on 0, 1, 2, 3, 4, 5, 6, 7, 14 and 21 DPI. Samples were always obtained prior to dosing with mock or peptide. Swabs were stored at −80° C. in virus transport medium (Minimum Essential Medium Eagle with Hank's BSS (Lonza), 5 g/L lactalbumine enzymatic hydrolysate, 10% glycerol (Sigma-Aldrich), 200 U/ml of penicillin, 200 mg/ml of streptomycin, 100 U/ml of polymyxin B sulfate (Sigma-Aldrich), and 250 mg/ml of gentamicin (Life Technologies). Blood samples were obtained from ferrets on 0, 7, 14 and 21 DPI by vena cava puncture. Blood was collected in serum-separating tubes (Greiner), processed, heat-inactivated and sera were stored at −80° C.

To assess susceptibility of ferrets to SARS-COV-2 post-treatment, previously mock-treated or [SARS_{HRC}-PEG₄]₂-chol-treated ferrets were re-housed in pairs of the same treatment schedule into six isolators. Ferrets were challenged in a titration fashion with 5×10³, 5×10⁴ or 5×10⁵ TCID₅₀/ml (in 450 μl) of SARS-COV-2. For each dose, two mock-treated and two peptide-treated ferrets were inoculated intranasally. Daily throat and nose swabs were collected from the animals until day 7. All susceptible animals were productively infected with SARS-COV-2 in a dose-dependent manner (FIG. 23).

A second ferret transmission experiment (FIG. 24) was conducted similarly to the first experiment with the following modifications: [1] three donor ferrets were inoculated with 4×10⁵ TCID₅₀/ml (in 450 μl). [2] Two hours prior to cohousing of donor animals and direct contact animals, six contact animals were treated with a single dose of [SARS_{HRC}-PEG₄]₂-chol formulated in sucrose at an intended dose of 5 mg/kg. Leftover batches were stored at −80° ° C. for later testing in in vitro potency assays, at which stage we observed that IC₅₀ of the lipopeptide formulated in sucrose (prepared at 10 mg scale instead of the 1 mg scale shown in FIG. 16) was approximately 20-fold higher than intended (FIG. 22C,D). Therefore in this experiment the animals received a substantially lower dose than in the experiment with DMSO-formulated peptide. [3] The six mock-treated ferrets received a single dose of [HPIV3_{HRC}-PEG₄]₂-chol formulated in sucrose (5 mg/kg). At 3 DPI, 22 hours post cohousing, donor animals were moved back to their original isolator and [HPIV3_{HRC}-PEG₄]₂-chol-treated and [SARS_{HRC}-PEG₄]₂-chol-treated animals were housed in groups of six. Throat and nose swabs were collected from the animals on 0, 1, 2, 3, 4, 5, 6, and 7, blood was collected at 0 and 7 DPI. [SARS_{HRC}-PEG₄]₂-chol-treated animals were rehoused into a separate isolator upon detection of SARS-COV-2 in throat or nose swab (Ct<30). The experiment was stopped at 7 DPI.

All animal handling was performed under anesthesia with a mixture of ketamine/medetomidine (10 mg/kg and 0.05 mg/kg, respectively) antagonized by atipamezole (0.25 mg/kg). All animal experiments were performed in class III isolators in a negatively pressurized ABSL3 facility. Ferrets were weighed on a daily basis. Body weights of all ferrets remained stable over time in both the experiment with DMSO-formulated peptides and sucrose-formulated peptides (FIG. 25).

RNA isolation and RT-qPCR on throat and nose swabs. Sixty μl of sample (virus transport medium in which swabs are stored) was added to 90 μl of MagNA Pure 96 External Lysis Buffer (Roche). A known concentration of phocine distemper virus (PDV) was added to the sample as internal control for the RNA extraction. The 150 μl of sample/lysis buffer was added to a well of a 96-well plate containing 50 μl of magnetic beads (AMPure XP, Beckman Coulter). After thorough mixing, the plate was incubated for 15 min at room temperature. The plate was then placed on a magnetic block (DynaMag™-96 Side Skirted Magnet (ThermoFisher Scientific)) and incubated for 3 min to allow the displacement of the beads towards the side of the magnet. Supernatants were carefully removed and beads were washed three times for 30 sec at room temperature with 200 μl/well of 70% ethanol. After the last wash, a 20 μl multi-channel pipet was used to remove residual ethanol. Plates were air-dried for 2 min at room temperature. Plates were removed from the magnetic block and 50 μl of PCR grade water was added to each well and mixed. Plates were incubated for 5 min at room temperature and then placed back on the magnetic block for 2 min to allow separation of the beads. Supernatants were pipetted in a new plate and RNA was stored at −80° C. The RNA was directly used for RT-qPCR using primers and probes targeting the E gene of SARS-COV-2 as previously described.

Virus isolation from throat and nose swabs. SARS-COV-2 was isolated in VeroE6 using an infectious center assay determining the tissue culture infectious dose-50 (TCID₅₀/ml). Cells were inoculated with 50 μl of sample (virus transport medium in which swabs are stored, first dilution 1:3), which was diluted in a 3-fold dilution series in quadruplicate. VeroE6 were screened for cytopathic effect (CPE) after 6 days of culture, the infectious titer in TCID₅₀/ml was calculated.

Detection of SARS-COV-2-specific antibodies in ferret sera. Seroconversion of ferrets was tested with ferret sera obtained at 0, 7, 14 and 21 DPI. A S and nucleocapsid (N) ELISA were performed. High-binding ELISA plates were coated with 20 ng recombinant His-tagged S protein (SinoBiological) or 100 ng recombinant His-tagged N protein (SinoBiological) in PBS overnight at 4° C. Subsequently, plates were washed with PBS-Tween followed by a blocking step with Blocker blotto in TBS (Life technologies) containing 0.01% Tween-20 (37° ° C., 1 hr). Sera were tested in duplicate at a concentration of 1:100 diluted in blocking buffer. After 1 hr incubation at 37° C., plates were washed and incubated with goat-anti-ferret IgG H&L/HRP (Abcam) for 1 h at 37° C. After washing, TMB substrate (Seracare) was incubated for 5 minutes in the dark. The reaction was stopped using sulfuric acid and absorbance was measured at 450 nm in a Tecan M200 plate reader. Virus neutralizing antibodies were detected by endpoint titration assay. Briefly, duplicates of ferret sera were incubated with 100 TCID₅₀ of SARS-COV-2 in a 2-fold dilution series starting at a concentration of 1:8 for 1 hr at 37° C. Virus-sera mix was added to VeroE6 cells and incubated for 5 days at 37° C. CPE was used as readout to determine the minimal serum concentration required to inhibit CPE.

Statistics. Inhibitory concentrations 50% and 90% (IC₅₀ and IC₉₀, respectively) in fusion assays were calculated by performing three parameter nonlinear regression. The difference between IC₅₀s were compared by Two-way ANOVA. IC₅₀ and IC₉₀ in infectious virus assays were calculated by performing four parameter nonlinear regression with variable slope on normalized and transformed data. All line graphs in in vivo experiments were compared by 2-way ANOVA repeated measures. Areas under the curve were calculated with GraphPad Prism on basis of curves per animal, and were compared by Mann-Whitney test. All statistics were performed with GraphPad Prism V9.

### Example 8: In Vivo Potency Test of [SARS-PEG4]2-Chol and SARS_{HRC}-PEG₂₄-Chol in a Transgenic Mouse Model

SARS-COV-2 is a Betacoronavirus that emerged from China in 2019. It is responsible for the COVID-19 (Coronavirus disease 2019) pandemic that has already caused millions of deaths worldwide. Although vaccines are available, it is important to have alternative and complementary prophylaxis, especially for people who are vulnerable or refractory to vaccination.

The entry of SARS-COV-2 occurs through the attachment and fusion of the viral envelope with the plasma membrane of the host cell and is mediated by the viral glycoprotein S. This trimeric class I protein has N- and C-terminal repeated heptades (HR) organized in 6 anti-parallel helixes. We generated peptides from the HR region at the C-terminal position of the S protein of SARS-COV-2, coupled to a lipid. These peptides inhibit viral entry by binding the N-terminal heptad-repeat (HR) regions of the surface proteins. We tested their ability to inhibit viral entry into the cell both in vitro and ex vivo and to prevent viral transmission. The ability of these peptides to protect an animal model sensitive to SARS-CoV-2 was also investigated through an in vivo study.

Certain peptides inhibit viral fusion with a 90% inhibitory concentration (IC90) in the nanomolar range, and subsequently infection and viral dissemination in mice. These peptides were then administered to transgenic mice expressing the human ACE2 receptor, under the control of cytokeratin K18 (B6.Cg-Tg (K18-ACE2) 2Prlmn/J, Jackson) promoter intranasally, prior to infection with SARS-COV-2. Although infection was generally 100% lethal within 10 days post-infection in K18-hACE2 mice, 80-100% of animals treated with these 2 peptides survived respectively, with significantly reduced viral loads in the lungs 2 days post-infection, compared to untreated animals.

In conclusion, these results demonstrate that the peptides that inhibit fusion between the virus and its host cell also block the respiratory infection of SARS-COV-2 in vivo in a mouse model, thus constituting a new antiviral approach to be developed through administration of the inhibitory peptides, in order to fight the current covid-19 pandemic.

Sequence Listing

SEQ ID NO: 1 (SARS Peptide, also named SARS_{HRC})
DISGINASVVNIQKEIDRLNEVAKNLNESLIDLQEL
SEQ ID NO: 2 (SARSMod Peptide)
DISQINASVVNIEYEIKKLEEVAKKLEESLIDLQEL
SEQ ID NO: 3 (>sp|P0DTC2|SPIKE_SARS2 Spike glycoprotein OS = Severe acute)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRENGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKG
VKLHYT

### REFERENCES

Li, F. Structure, Function, and Evolution of Coronavirus Spike Proteins. Annu Rev Virol 3, 237-261, doi: 10.1146/annurev-virology-110615-042301 (2016).
Hoffmann, M. et al. SARS-COV-2 Cell Entry Depends on ACE2 and TMPRSS2 and Is Blocked by a Clinically Proven Protease Inhibitor. Cell, doi:10.1016/j.cell.2020.02.052 (2020).
Wan, Y., Shang, J., Graham, R., Baric, R. S. & Li, F. Receptor Recognition by the Novel Coronavirus from Wuhan: an Analysis Based on Decade-Long Structural Studies of SARS Coronavirus. J Virol 94, doi: 10.1128/JVI.00127-20 (2020).
Bosch, B. J., van der Zee, R., de Haan, C. A. & Rottier, P. J. The coronavirus spike protein is a class I virus fusion protein: structural and functional characterization of the fusion core complex. J Virol 77, 8801-8811, doi: 10.1128/jvi.77.16.8801-8811.2003 (2003).
Outlaw, V. K. et al. Inhibition of Coronavirus Entry In Vitro and Ex Vivo by a Lipid-Conjugated Peptide Derived from the SARS-COV-2 Spike Glycoprotein HRC Domain. mBio 11, doi: 10.1128/mBio.01935-20 (2020).
Xia, S., Wang, Q., Liu, S. W., Lu, L. & Jiang, S. B. [Development of peptidic MERS-COV entry inhibitors]. Yao Xue Xue Bao 50, 1513-1519 (2015).
Xia, S. et al. A pan-coronavirus fusion inhibitor targeting the HR1 domain of human coronavirus spike. Sci Adv 5, eaav4580, doi: 10.1126/sciadv.aav4580 (2019).
Xia, S. et al. Inhibition of SARS-COV-2 (previously 2019-nCOV) infection by a highly potent pan-coronavirus fusion inhibitor targeting its spike protein that harbors a high capacity to mediate membrane fusion. Cell Res 30, 343-355, doi: 10.1038/s41422-020-0305-x (2020).
Zhu, Y., Yu, D., Yan, H., Chong, H. & He, Y. Design of Potent Membrane Fusion Inhibitors against SARS-COV-2, an Emerging Coronavirus with High Fusogenic Activity. J Virol 94, doi: 10.1128/JVI.00635-20 (2020).
Wang, X. et al. Broad-Spectrum Coronavirus Fusion Inhibitors to Combat COVID-19 and Other Emerging Coronavirus Diseases. Int J Mol Sci 21, doi: 10.3390/ijms21113843 (2020).
Porotto, M. et al. Viral entry inhibitors targeted to the membrane site of action. J Virol 84, 6760-6768, doi: 10.1128/JVI.00135-10 (2010).
Porotto, M. et al. Inhibition of Nipah virus infection in vivo: targeting an early stage of paramyxovirus fusion activation during viral entry. PLOS Pathog 6, e1001168, doi:10.1371/journal.ppat.1001168 (2010).
Pessi, A. et al. A general strategy to endow natural fusion-protein-derived peptides with potent antiviral activity. PLOS One 7, e36833, doi: 10.1371/journal.pone.0036833 (2012).
Welsch, J. C. et al. Fatal measles virus infection prevented by brain-penetrant fusion inhibitors. J Virol 87, 13785-13794, doi: 10.1128/JVI.02436-13 (2013).
Outlaw, V. K. et al. Dual Inhibition of Human Parainfluenza Type 3 and Respiratory Syncytial Virus Infectivity with a Single Agent. J Am Chem Soc 141, 12648-12656, doi:10.1021/jacs.9b04615 (2019).
Figueira, T. N. et al. Structure-Stability-Function Mechanistic Links in the Anti-Measles Virus Action of Tocopherol-Derivatized Peptide Nanoparticles. ACS Nano 12, 9855-9865, doi: 10.1021/acsnano.8b01422 (2018).
Figueira, T. N. et al. Effective in Vivo Targeting of Influenza Virus through a Cell-Penetrating/Fusion Inhibitor Tandem Peptide Anchored to the Plasma Membrane. Bioconjug Chem 29, 3362-3376, doi: 10.1021/acs.bioconjchem.8b00527 (2018).
Mathieu, C. et al. Broad spectrum antiviral activity for paramyxoviruses is modulated by biophysical properties of fusion inhibitory peptides. Sci Rep 7, 43610, doi:10.1038/srep43610 (2017).
Figueira, T. N. et al. In Vivo Efficacy of Measles Virus Fusion Protein-Derived Peptides Is Modulated by the Properties of Self-Assembly and Membrane Residence. J Virol 91, doi:10.1128/JVI.01554-16 (2017).
Mathieu, C. et al. Prevention of measles virus infection by intranasal delivery of fusion inhibitor peptides. J Virol 89, 1143-1155, doi: 10.1128/JVI.02417-14 (2015).
Zhang, L. et al. The D614G mutation in the SARS-COV-2 spike protein reduces S1 shedding and increases infectivity. bioRxiv, doi: 10.1101/2020.06.12.148726 (2020).
Muik, A. et al., Neutralization of SARS-COV-2 lineage B.1.1.7 pseudovirus by BNT162b2 228 vaccine-elicited human sera. Science, (2021).
Wu K. et al., mRNA-1273 vaccine induces neutralizing antibodies against spike mutants from 230 global SARS-COV-2 variants. bioRxiv, (2021)
Mykytyn, A. Z. et al. The SARS-COV-2 multibasic cleavage site facilitates early serine protease-mediated entry into organoid-derived human airway cells. bioRxiv, 2020.2009.2007.286120, doi:10.1101/2020.09.07.286120 (2020).
Hoffmann, M. et al. Chloroquine does not inhibit infection of human lung cells with SARS-CoV-2. Nature 585, 588-590, doi: 10.1038/s41586-020-2575-3 (2020).
Richard, M. et al. SARS-COV-2 is transmitted via contact and via the air between ferrets. Nat Commun 11, 3496, doi: 10.1038/s41467-020-17367-2 (2020).
Munster, V. J. et al. Pathogenesis and transmission of swine-origin 2009 A(H1N1) influenza virus in ferrets. Science 325, 481-483, doi: 10.1126/science. 1177127 (2009).
Martina, B. E. et al. Virology: SARS virus infection of cats and ferrets. Nature 425, 915, doi:10.1038/425915a (2003).
Kim, Y. I. et al. Infection and Rapid Transmission of SARS-COV-2 in Ferrets. Cell Host Microbe, doi:10.1016/j.chom.2020.03.023 (2020).
Cao, L. et al. De novo design of picomolar SARS-COV-2 miniprotein inhibitors. Science 370, 426-431, doi: 10.1126/science.abd9909 (2020).
Schoof, M. et al. An ultra-high affinity synthetic nanobody blocks SARS-COV-2 infection by locking Spike into an inactive conformation. bioRxiv, doi: 10.1101/2020.08.08.238469 (2020).
Schoof, M. et al. An ultrapotent synthetic nanobody neutralizes SARS-COV-2 by stabilizing inactive Spike. Science 370, 1473-1479, doi: 10.1126/science.abe3255 (2020).
Cox, R. M., Wolf, J. D. & Plemper, R. K. Therapeutically administered ribonucleoside analogue MK-4482/EIDD-2801 blocks SARS-COV-2 transmission in ferrets. Nat Microbiol, doi:10.1038/s41564-020-00835-2 (2020).
T. A. Halgren, B. L. Bush, The Merck molecular force field (MMFF94). Extension and application. Abstr Pap Am Chem S 212, 2-Comp (1996).
R. B. Best et al., Optimization of the Additive CHARMM All-Atom Protein Force Field Targeting Improved Sampling of the Backbone phi, psi and Side-Chain chi(1) and chi(2) Dihedral Angles. J Chem Theory Comput 8, 3257-3273 (2012).
S. J. Marrink, H. J. Risselada, S. Yefimov, D. P. Tieleman, A. H. de Vries, The MARTINI force field: Coarse grained model for biomolecular simulations. J Phys Chem B 111, 7812-7824 (2007).
Y. Cai et al., Distinct conformational states of SARS-COV-2 spike protein. Science 369, 1586-1592 (2020).
S. Hakansson-McReynolds, S. Jiang, L. Rong, M. Caffrey, Solution structure of the severe acute respiratory syndrome-coronavirus heptad repeat 2 domain in the prefusion state. J Biol Chem 281, 11965-11971 (2006).
T. N. Figueira et al., In Vivo Efficacy of Measles Virus Fusion Protein-Derived Peptides Is Modulated by the Properties of Self-Assembly and Membrane Residence. J Virol 91(1). pii: e01554-16. (2016).
A. Z. Mykytyn et al., The SARS-COV-2 multibasic cleavage site facilitates early serine protease-mediated entry into organoid-derived human airway cells. bioRxiv, 2020.2009.2007.286120 (2020).
M. Porotto et al., Inhibition of Nipah virus infection in vivo: targeting an early stage of paramyxovirus fusion activation during viral entry. PLOS Pathog 6, e1001168 (2010).
V. K. Outlaw et al., Inhibition of Coronavirus Entry In Vitro and Ex Vivo by a Lipid-Conjugated Peptide Derived from the SARS-COV-2 Spike Glycoprotein HRC Domain. mBio 11(5):e01935-20. (2020)
G. J. van Doornum, M. Schutten, J. Voermans, G. J. Guldemeester, H. G. Niesters, Development and implementation of real-time nucleic acid amplification for the detection of enterovirus infections in comparison to rapid culture of various clinical specimens. J Med Virol 79, 1868-1876 (2007).
V. M. Corman et al., Detection of 2019 novel coronavirus (2019-nCOV) by real-time RT-PCR. Euro Surveill 25 (3):2000045 (2020).
J. Park and T. Gallagher, Lipidation increases antiviral activities of coronavirus fusion-inhibiting peptides, Virology 2017; 511, 9-18

1-56. (canceled)

57. A SARS-COV2 (COVID-19) lipid-peptide fusion inhibitor comprising two peptides each comprising SEQ ID NO:1, two PEG moieties, and one cholesterol tag.

58. The SARS-COV2 (COVID-19) lipid-peptide fusion inhibitor of claim 57, wherein each of the two peptides comprises SEQ ID NO:5.

59. The SARS-COV2 (COVID-19) lipid-peptide fusion inhibitor of claim 57, wherein each of the two PEG moieties is a PEG4 moiety.

60. The SARS-COV2 (COVID-19) lipid-peptide fusion inhibitor of claim 58, wherein each of the two PEG moieties is a PEG4 moiety.

61. The SARS-COV2 (COVID-19) lipid-peptide fusion inhibitor of claim 60, having a structure as shown in FIG. 11A.

62. A SARS-COV2 (COVID-19) lipid-peptide fusion inhibitor comprising two peptides each comprising SEQ ID NO:1, one cholesterol tag, and means for linking the two peptides to the cholesterol tag.

63. The SARS-COV2 (COVID-19) lipid-peptide fusion inhibitor of claim 62, wherein each of the two peptides comprises SEQ ID NO:5.

64. A pharmaceutical composition comprising the SARS-COV2 (COVID-19) lipid-peptide fusion inhibitor of claim 57 and a pharmaceutically acceptable excipient.

65. A method of preventing COVID-19 in a subject in need thereof, comprising administering the pharmaceutical composition of claim 64 to the subject.

66. A method of reducing the risk of COVID-19 in a subject in need thereof, comprising administering the pharmaceutical composition of claim 64 to the subject.

67. A method of reducing the risk of death from COVID-19 in a subject in need thereof, comprising administering the pharmaceutical composition of claim 64 to the subject.

68. A method of reducing the risk of SARS-COV-2 infecting a cell in a subject, comprising administering the pharmaceutical composition of claim 64 to the subject.

69. A method of treating a subject having COVID-19, comprising administering the pharmaceutical composition of claim 64 to the subject.

70. The method of claim 65, wherein the pharmaceutical composition is administered per airway.

71. The method of claim 70, wherein the pharmaceutical composition is administered intranasally.

72. The method of claim 70, wherein the pharmaceutical composition is administered as nasal drops or a spray.

73. The method of claim 70, wherein the pharmaceutical composition is administered as a nasal powder.

74. The method of claim 65, wherein the pharmaceutical composition is administered to the subject at least two times.

75. The method of claim 74, wherein the administration occurs daily.

76. The method of claim 65, wherein the pharmaceutical composition is administered to the subject at least one time before the subject is exposed to SARS-COV-2.


SEQUENCE LISTING (13 sequences)

SEQ ID NO: 1 - protein, 36 aa, Severe acute respiratory syndrome coronavirus 2
DISGINASVVNIQKEIDRLNEVAKNLNESLIDLQEL

SEQ ID NO: 2 - protein, 36 aa, Artificial Sequence
  Description of Artificial Sequence Synthetic polypeptide
DISQINASVVNIEYEIKKLEEVAKKLEESLIDLQEL

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 5 aa, Artificial Sequence
  Description of Artificial Sequence Synthetic peptide
GSGSC

SEQ ID NO: 5 - protein, 42 aa, Artificial Sequence
  Description of Artificial Sequence Synthetic polypeptide
DISGINASVVNIQKEIDRLNEVAKNLNESLIDLQELGSGSGC

SEQ ID NO: 6 - protein, 42 aa, Artificial Sequence
  Description of Artificial Sequence Synthetic polypeptide
DISQINASVVNIEYEIKKLEEVAKKLEESLIDLQELGSGSGC

SEQ ID NO: 7 - protein, 41 aa, Artificial Sequence
  Description of Artificial Sequence Synthetic polypeptide
DISGINASVVNIQKEIDRLNEVAKNLNESLIDLQELGSGSG

SEQ ID NO: 8 - protein, 36 aa, Artificial Sequence
  Description of Artificial Sequence Synthetic polypeptide
FNVALDQVFESIENSQALVDQSNRILSSAEKGSGSG

SEQ ID NO: 9 - protein, 35 aa, Artificial Sequence
  Description of Artificial Sequence Synthetic polypeptide
SYLNISDFRNDWILESDFLISEMLSKEYSDGSGSG

SEQ ID NO: 10 - protein, 27 aa, Artificial Sequence
  Description of Artificial Sequence Synthetic peptide
AWDFGSVGGVFTSVGKAVHQVFGSGSG

SEQ ID NO: 11 - protein, 41 aa, Artificial Sequence
  Description of Artificial Sequence Synthetic polypeptide
VALDPIDISIVLNKIKSDLEESKEWIRRSNKILDSIGSGSG

SEQ ID NO: 12 - protein, 4 aa, Artificial Sequence
  Description of Artificial Sequence Synthetic peptide
GSGS

SEQ ID NO: 13 - protein, 6 aa, Artificial Sequence
  Description of Artificial Sequence Synthetic peptide
GSGSGC